(19) United States
(12) Patent Application Publication
(10) Pub. No.: US 2016/0185763 A1
(43) Pub. Date: Jun. 30, 2016

(54) LIQUID CRYSTAL STABILIZER, LIQUID CRYSTAL COMPOSITION INCLUDING THE SAME, AND LIQUID CRYSTAL DISPLAY INCLUDING THE SAME

(71) Applicant: SAMSUNG DISPLAY CO., LTD., Yongin-City (KR)

(72) Inventors: Si Heun KIM, Gyeonggi-do (KR); Fusayuki TAKESHITA, Seoul (KR); Ji Hong BAE, Gyeonggi-do (KR); Beom-Soo SHIN, Gyeonggi-do (KR); Keun Chan OH, Chungcheongnam-do (KR); Hye Lim JANG, Gyeonggi-do (KR)

(21) Appl. No.: 14/744,584

(22) Filed: Jun. 19, 2015

(30) Foreign Application Priority Data
Dec. 29, 2014 (KR) 10-2014-0192263

(51) Int. Cl. C07D 405/08; C09K 19/54; G02F 1/1333; C07D 211/94; G02F 1/1368; C09K 19/30; C07D 211/46

(52) U.S. Cl. CPC C07D 405/08; C09K 19/3003; C09K 19/54; C07D 211/46; C07D 211/94; G02F 1/1368; G02F 1/133345; C09K 2019/3004; C09K 2019/301; C09K 2019/3019; C09K 2019/3006

(57) ABSTRACT

A liquid crystal stabilizer according to an exemplary embodiment of the present invention includes at least one of the compounds represented by the following Chemical Formulae 1 and 2.

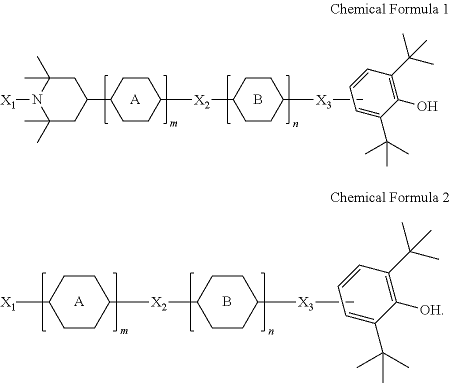

X₁ is any one of H, O, OR, and R, X₂ is any one of O, OCO, OR, RO, NOR, and R, X₃ is any one of O, OCO, OR, RO, NOR, and R, A and B each independently include one or more of a cyclohexyl group, a cyclic ether group, and a phenyl group, R is an alkyl group having 1 to 5 carbon atoms, and m and n are each independently a natural number from 0 to 2.

### CROSS-REFERENCE TO RELATED APPLICATION

This application claims priority under 35 U.S.C. §119 to Korean Patent Application No. 10-2014-0192263 filed in the Korean Intellectual Property Office on Dec. 29, 2014, the disclosure of which is incorporated by reference herein in its entirety.

(a) Technical Field

Exemplary embodiments of the present invention relate to a liquid crystal stabilizer, and more particularly to a liquid crystal composition including the liquid crystal stabilizer, and a liquid crystal display including the liquid crystal stabilizer.

(b) Discussion of Related Art

Liquid crystal displays may be flat panel displays, and may include two display panels facing each other, and a liquid crystal layer disposed between the two display panels. A field generating electrode such as a pixel electrode and a common electrode may be disposed on at least one of the two display panels.

In the liquid crystal display, by applying a voltage to the field generating electrode to generate an electric field on the liquid crystal layer, alignment of liquid crystal molecules disposed on the liquid crystal layer may be determined and transmittance of light passing through the liquid crystal layer may be adjusted.

In the liquid crystal display, a liquid crystal composition may display a desired image by adjusting transmittance of light. The liquid crystal display may have various characteristics such as low voltage driving, a high voltage holding ratio (VHR), a wide viewing angle characteristic, a wide motion temperature range, and a high-speed response characteristic.

To increase the high-speed response characteristic of the liquid crystal display, research related to rotational viscosity, refractive index, and elastic coefficient of the liquid crystal composition is being performed. However, when the liquid crystal display includes an alkenyl group, a linear afterimage may occur and reliability of the liquid crystal display may be reduced.

### SUMMARY

Exemplary embodiments of the present invention provide a liquid crystal composition with increased reliability including a liquid crystal stabilizer having a novel structure, and a liquid crystal display including the liquid crystal stabilizer.

An exemplary embodiment of the present invention provides a liquid crystal stabilizer including at least one of the compounds represented by the following Chemical Formulae 1 and 2.

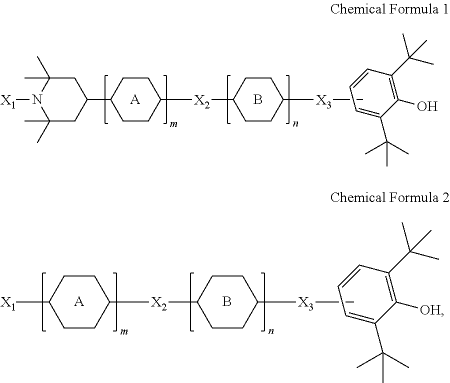

X₁ is any one of H, O, OR, and R, X₂ is any one of O, OCO, OR, RO, NOR, and R, X₃ is any one of O, OCO, OR, RO, NOR, and R, A and B each independently include one or more of a cyclohexyl group, a cyclic ether group, and a phenyl group, R is an alkyl group having 1 to 5 carbon atoms, and m and n are each independently a natural number from 0 to 2.

The compound represented by Chemical Formula 1 may be at least one of the compounds represented by the following Chemical Formulae 1-1 to 1-28.

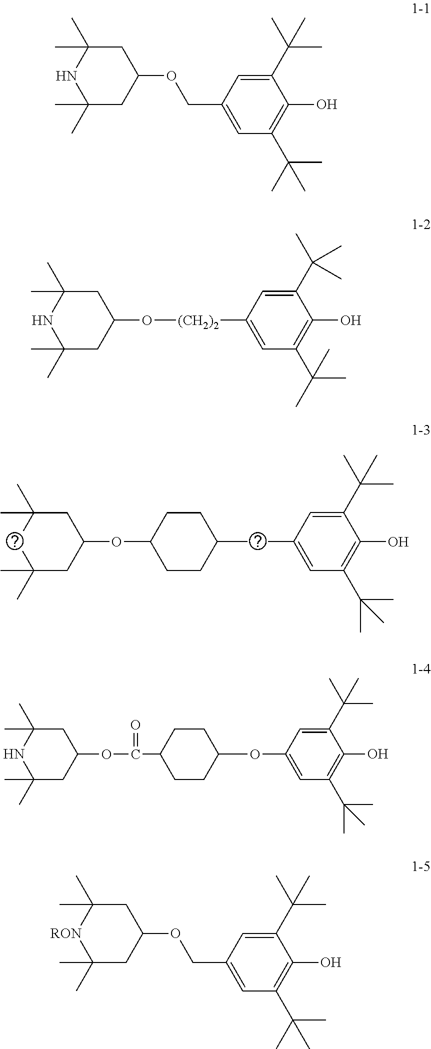

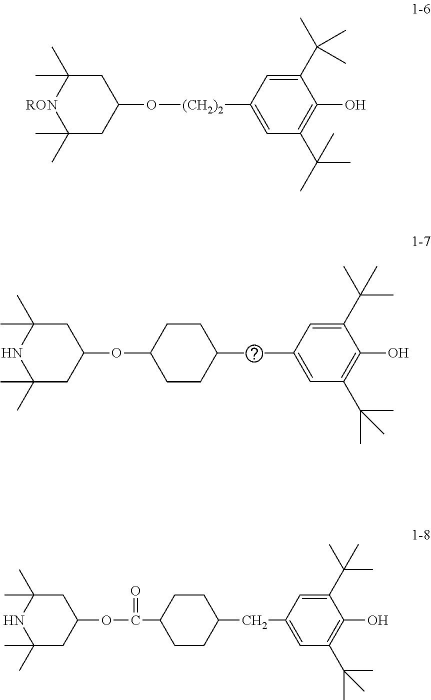

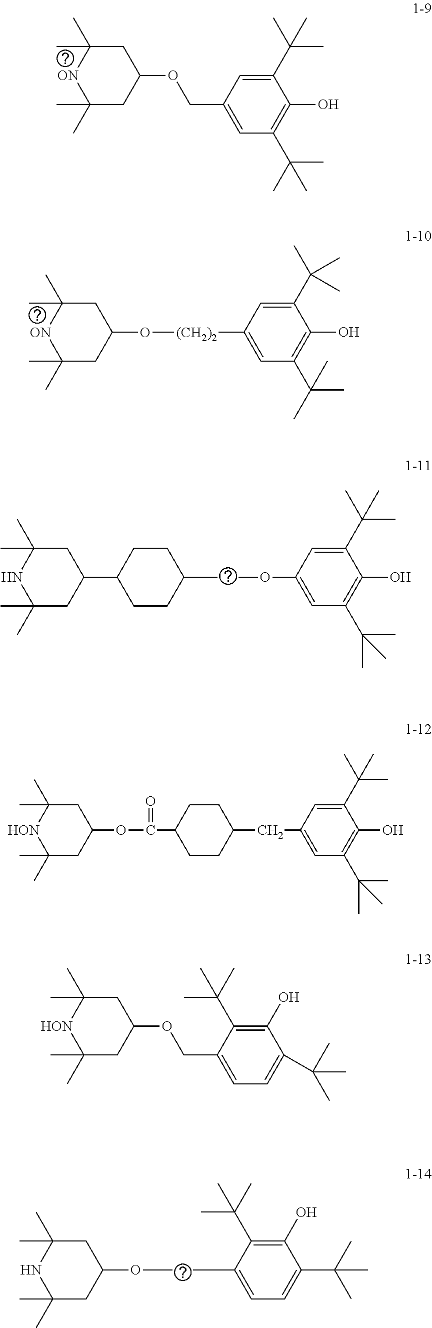

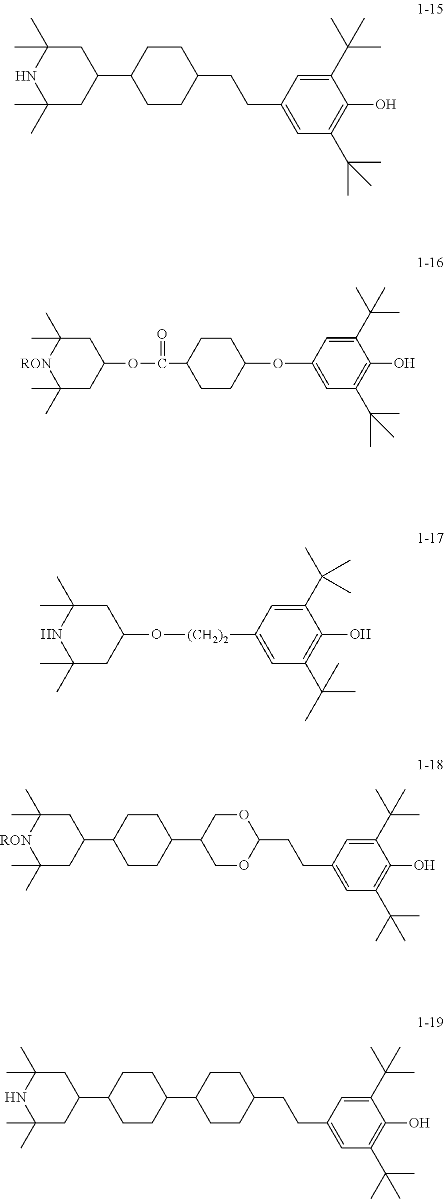

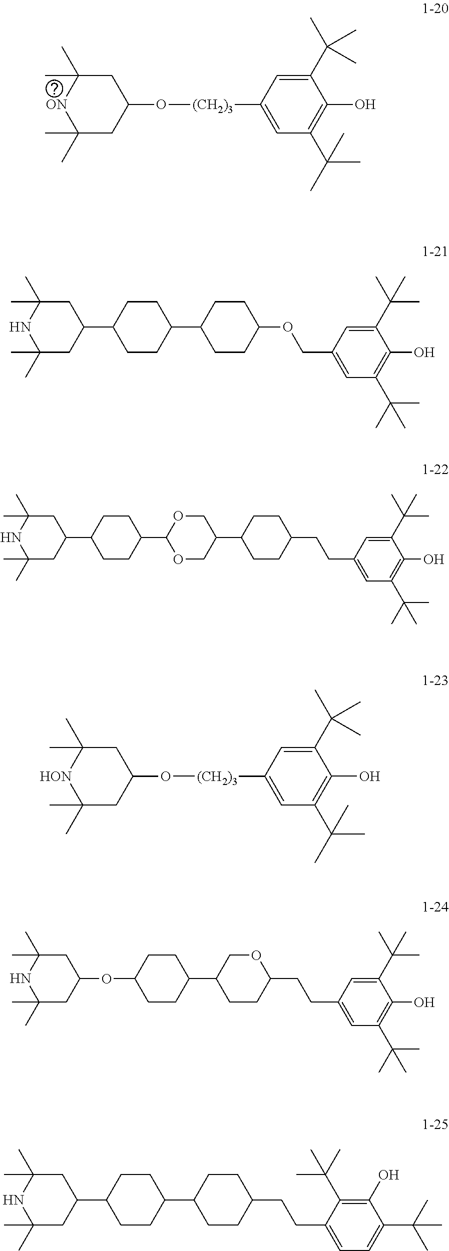

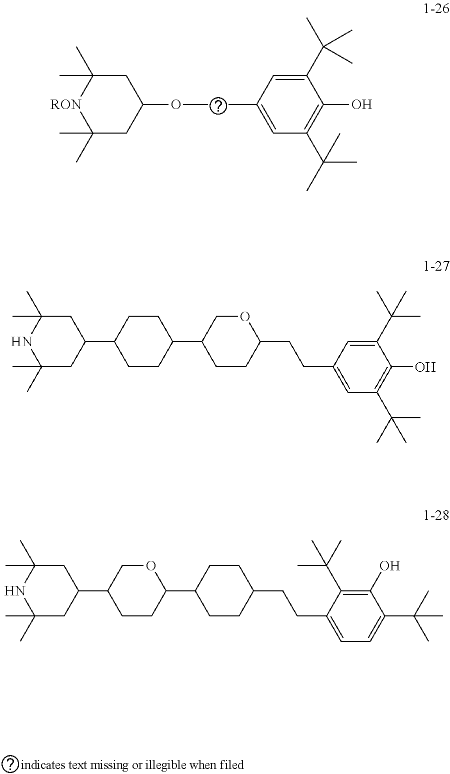

The compound represented by Chemical Formula 2 may be at least one of the compounds represented by the following Chemical Formulae 2-1 to 2-16.

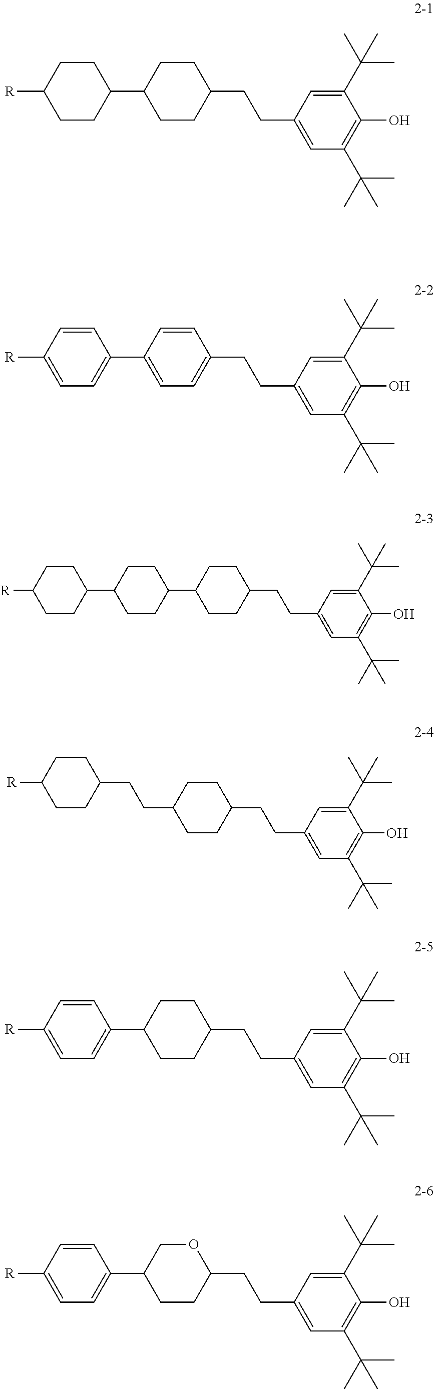

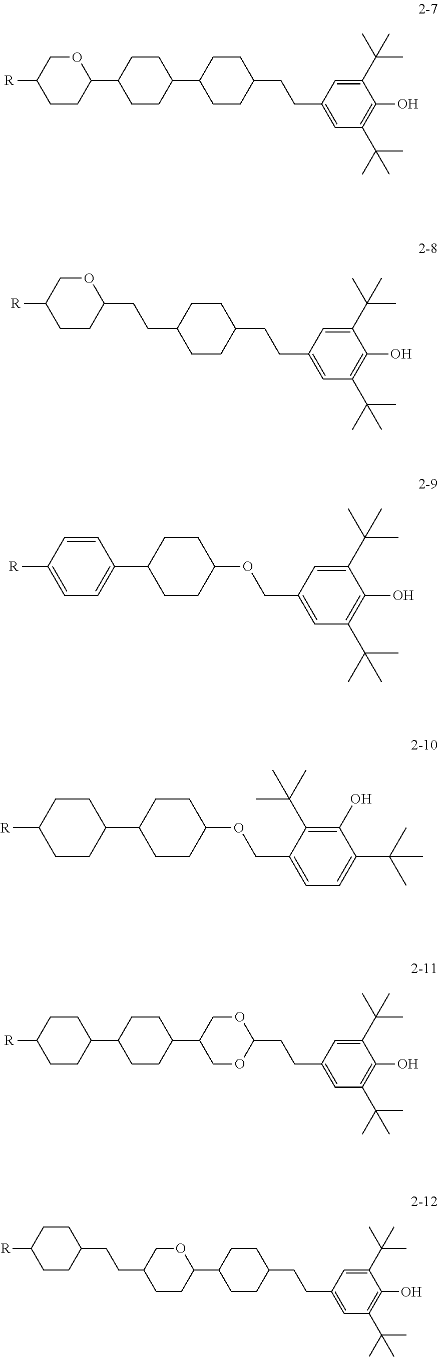

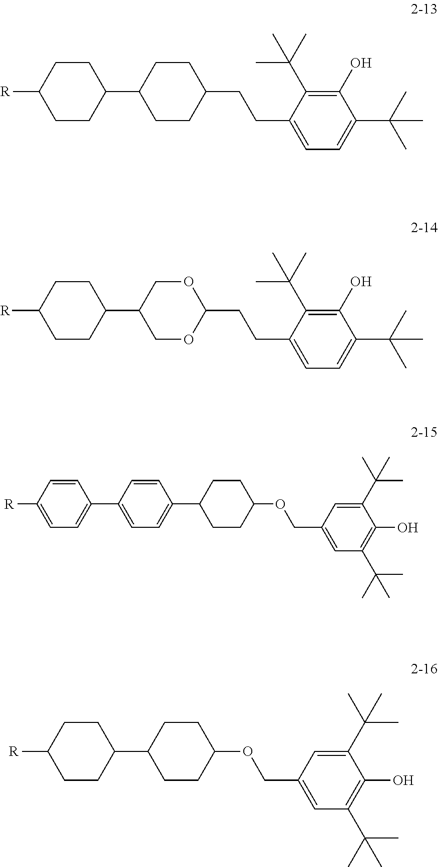

An exemplary embodiment of the present invention provides a liquid crystal composition including a first class of compounds including an alkenyl group, and a liquid crystal stabilizer including a compound represented by any one of the following Chemical Formulae 1 and 2.

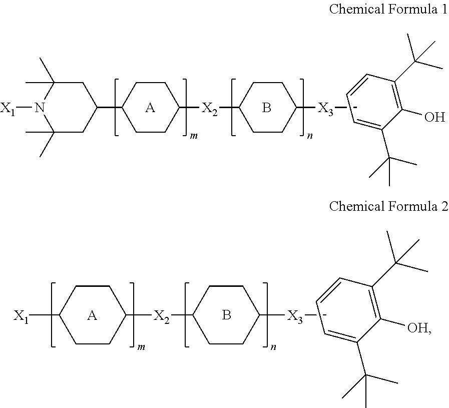

X₁ is any one of H, O, OR, and R, X₂ is any one of O, OCO, OR, RO, NOR, and R, X₃ is any one of O, OCO, OR, RO, NOR, and R, A and B each independently include one or more of a cyclohexyl group, a cyclic ether group, and a phenyl group, R is an alkyl group having 1 to 5 carbon atoms, and m and n are each independently a natural number from 0 to 2.

The liquid crystal composition may include the liquid crystal stabilizer in a content of from about 0.0001 wt % to about 10 wt % based on the liquid crystal composition.

The first class may include at least one of the compounds represented by the following Chemical Formulae 3-1 to 3-16.

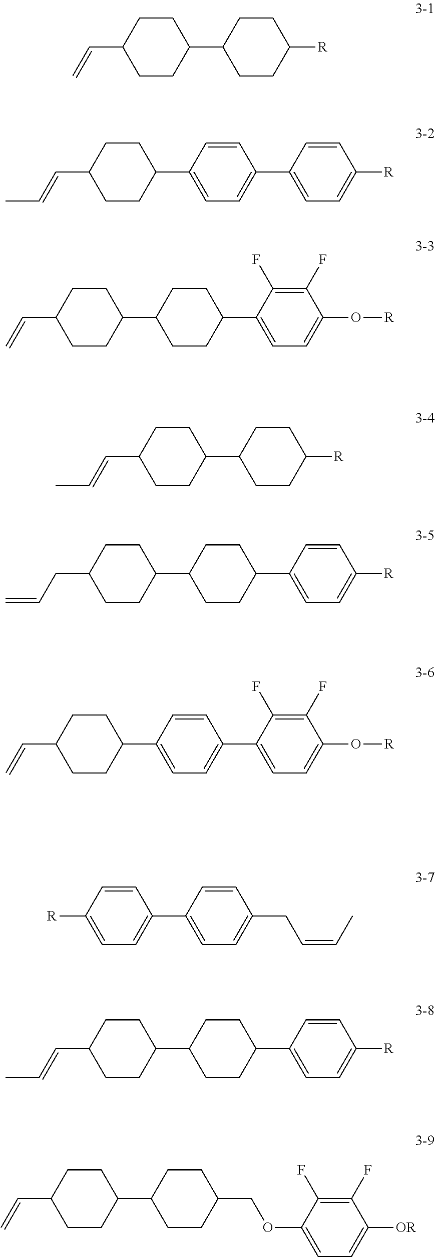

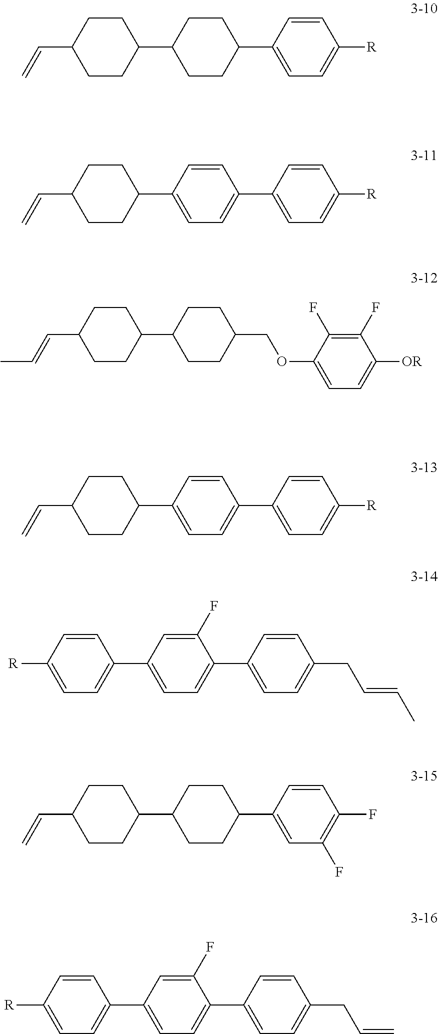

R is an alkyl group or an alkoxy group having 1 to 5 carbon atoms.

An exemplary embodiment of the present invention provides a liquid crystal display including a first insulation substrate. A thin film transistor is disposed on the first insulation substrate. A pixel electrode is disposed on the thin film transistor. A common electrode is disposed on the first insulation substrate. The common electrode is insulated from the pixel electrode. A second insulation substrate faces the first insulation substrate and is spaced apart from the first insulation substrate. A liquid crystal layer is disposed between the first insulation substrate and the second insulation substrate. The liquid crystal layer includes a first class of compounds including an alkenyl group, and at least one liquid crystal stabilizer selected from the compounds represented by the following Chemical Formulae 1 and 2.

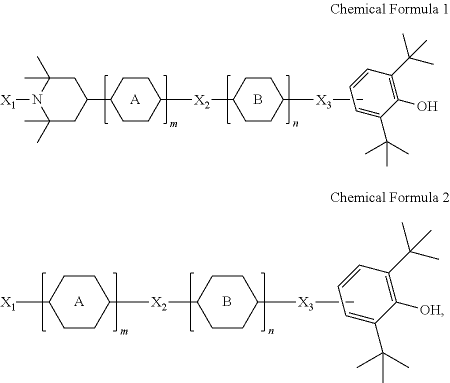

X₁ is any one of H, O, OR, and R, X₂ is any one of O, OCO, OR, RO, NOR, and R, X₃ is any one of O, OCO, OR, RO, NOR, and R, A and B each independently include one or more of a cyclohexyl group, a cyclic ether group, and a phenyl group, R is an alkyl group having 1 to 5 carbon atoms, and m and n are each independently a natural number from 0 to 2.

In the liquid crystal stabilizer and the liquid crystal composition including the liquid crystal stabilizer, an occurrence of impurities being formed in the liquid crystal composition may be reduced or prevented. Thus, an occurrence of afterimages and stains in the display panel may be reduced or prevented and thus a display device may be formed having higher reliability.

### BRIEF DESCRIPTION OF THE DRAWINGS

FIG. 1 is a top plan view of a pixel according to an exemplary embodiment of the present invention.

FIG. 2 is a cross-sectional view taken along line II-II of FIG. 1.

FIGS. 3 and 4 are voltage holding ratio graphs according to exemplary embodiments of the present invention and Comparative Examples.

FIGS. 5A to 5C are images showing whether there is an afterimage or not in exemplary embodiments of the present invention and the Comparative Examples.

### DETAILED DESCRIPTION OF THE EMBODIMENTS

Hereinafter, exemplary embodiments of the present invention will be described in more detail with reference to the accompanying drawings. Exemplary embodiments of the present invention may be modified in various different ways, all without departing from the spirit or scope of the present invention. In the drawings, the thickness of layers, films, panels, or regions may be exaggerated for clarity. It will be understood that when a layer is referred to as being “on” another layer or substrate, it may be directly on the other layer or substrate, or intervening them may be present. Like reference numerals may refer to like elements throughout the specification and drawings.

Hereinafter, a liquid crystal stabilizer according to an exemplary embodiment of the present invention will be described in more detail. The liquid crystal stabilizer may reduce or prevent a mechanism where liquid crystal molecules are activated by UV light or heat to react with radicals to form impurities.

The liquid crystal stabilizer according to an exemplary embodiment of the present invention may include at least one of the compounds represented by Chemical Formula 1 and Chemical Formula 2.

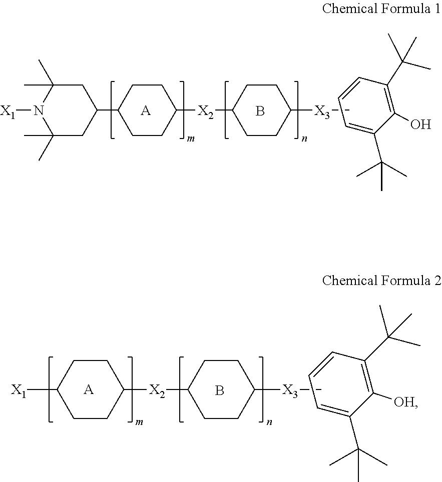

X₁ may be any one of H, O, OR, and R. X₂ may be any one of O, OCO, OR, RO, NOR, and R. X₃ may be any one of O, OCO, OR, RO, NOR, and R. A and B may each independently include one or more of a cyclohexyl group, a cyclic ether group, and a phenyl group. R may be an alkyl group having 1 to 5 carbon atoms, and m and n may each independently be a natural number of 0 to 2.

The compound represented by Chemical Formula 1 may have any structure satisfying the aforementioned condition, but for example, may be the same as that described below. X₃ may be at a para or meta position of a phenol group connected to a right side of Chemical Formula 1, and may include a cycloamine group connected to a left side of Chemical Formula 1.

The compound represented by Chemical Formula 1 may reduce or prevent an occurrence of a reaction of the liquid crystal molecules by exposure to UV light or heat, and thus may increase reliability of a liquid crystal composition including the compound represented by Chemical Formula 1.

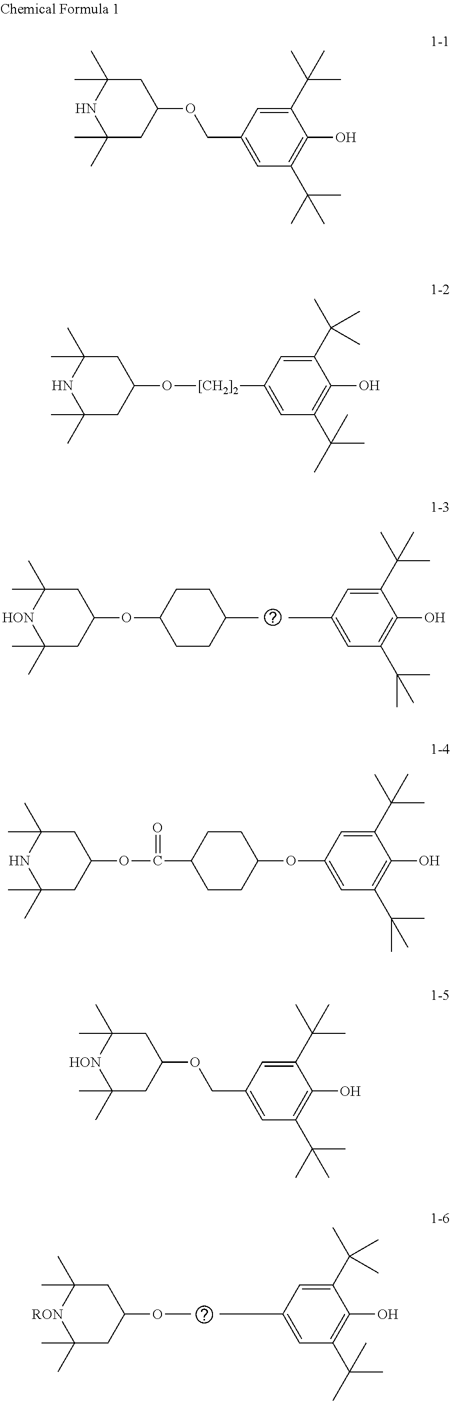

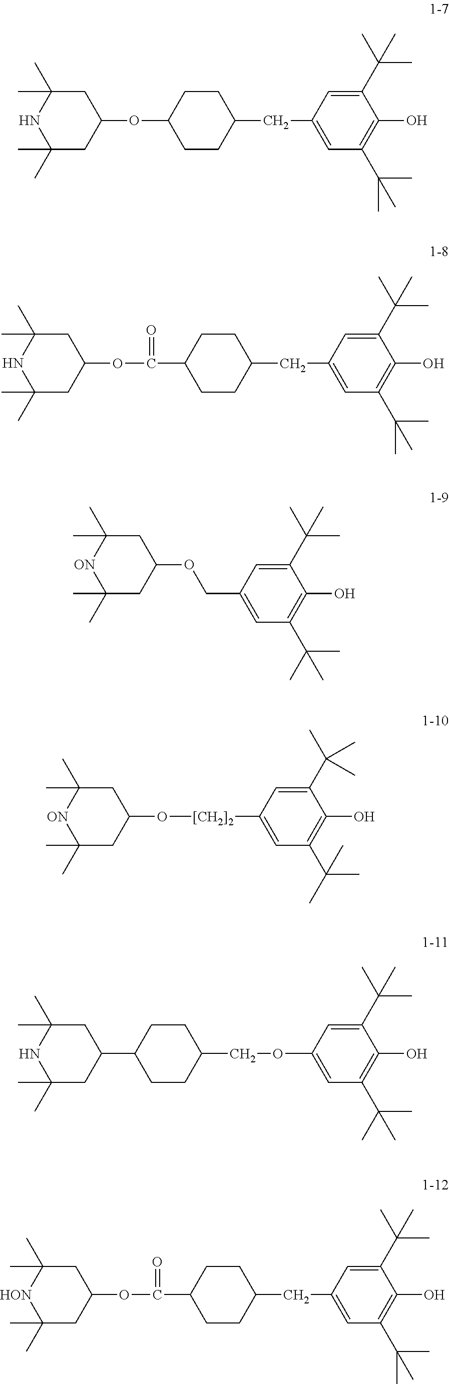

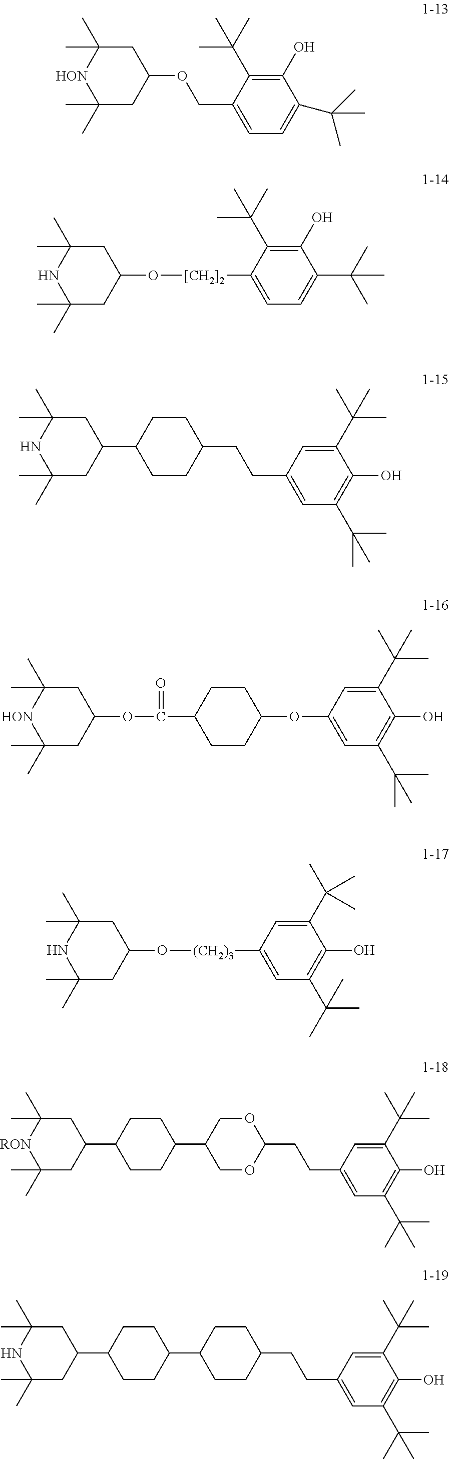

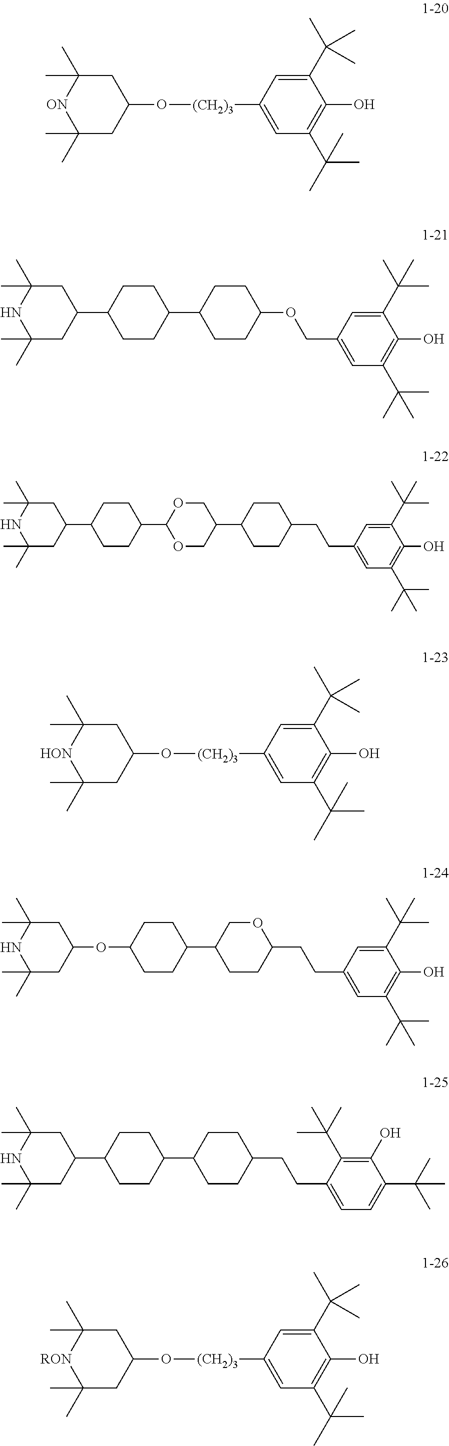

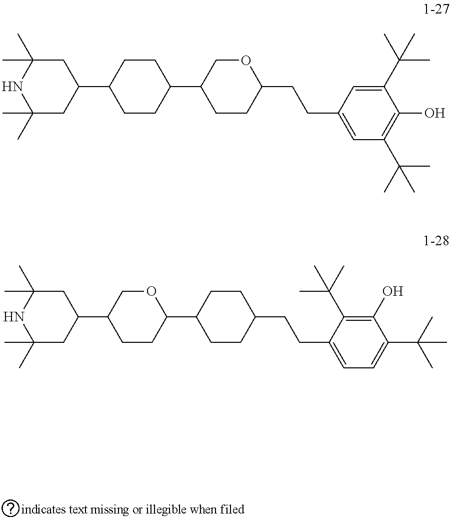 [SMILES: CC1(C)CC(C2CCC(C3CCC(CCc4cc(C(C)(C)C)c(O)c(C(C)(C)C)c4)OC3)CC2)CC(C)(C)N1 ; CC1(C)CC(C2CCC(C3CCC(CCc4ccc(C(C)(C)C)c(O)c4C(C)(C)C)CC3)OC2)CC(C)(C)N1]

The compound represented by Chemical Formula 2 may have any structure satisfying the aforementioned condition, but for example, may be the same as that described below. X₃ may be at a para or meta position of a phenol group connected to a right side of Chemical Formula 2.

The compound represented by Chemical Formula 2 may be connected through flexible X₃ (e.g., an alkyl group or alkoxy group), and thus may have an increased nematic characteristic. Further, the compound represented by Chemical Formula 2 may reduce or prevent an occurrence of the reaction of the liquid crystal molecules by exposure to UV light or heat, and thus may increase reliability of the liquid crystal composition.

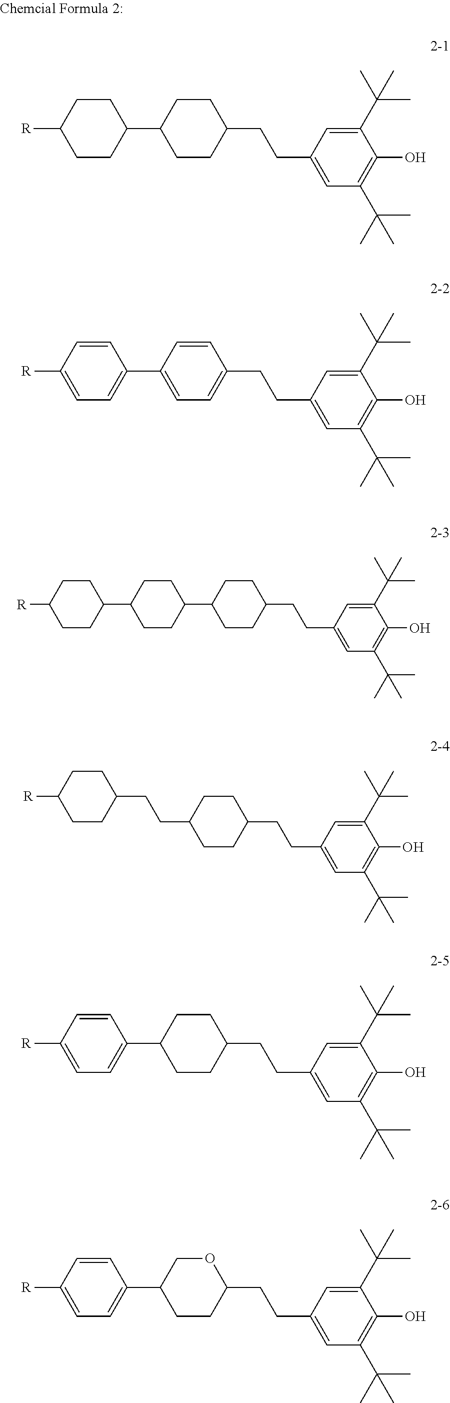

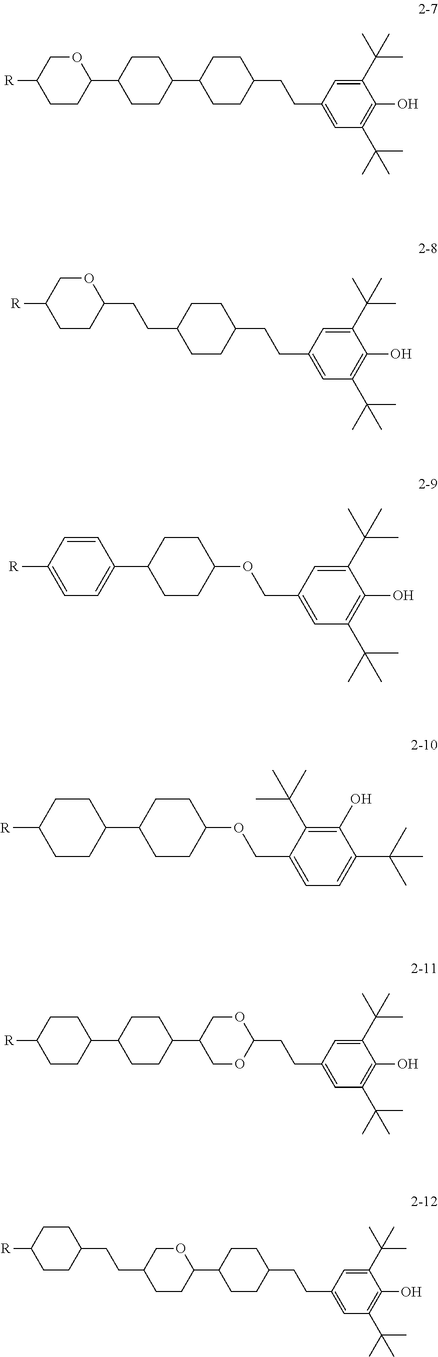

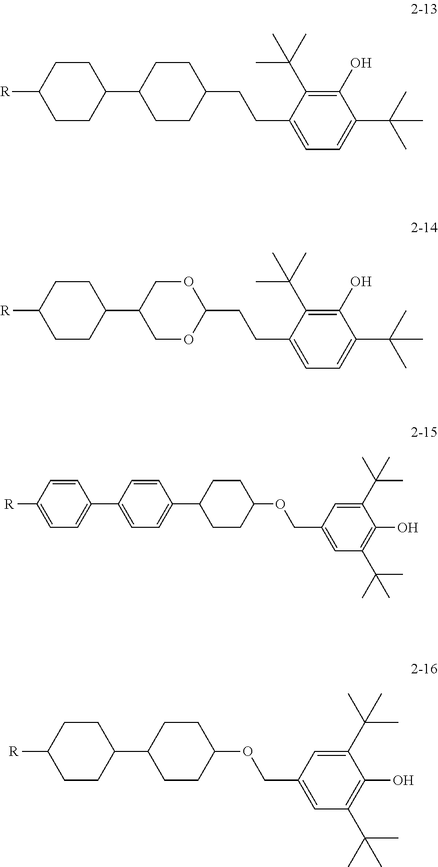

The liquid crystal stabilizer represented by the Chemical Formulae 1 and 2 may be injected into a liquid crystal layer of a display device, and may reduce or prevent the liquid crystal molecules from reacting with radicals by exposure to UV light or heat. When the reaction between the liquid crystal molecules and the free radicals is reduced or prevented, an occurrence of linear afterimages and/or stains in a display panel may be reduced or prevented.

Hereinafter, as an example of the compound represented by Chemical Formula 1, a synthesis of a compound represented by the following Chemical Formula 1-1 will be discussed in more detail.

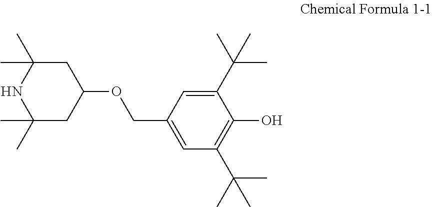 [SMILES: CC1(C)CC(OCc2cc(C(C)(C)C)c(O)c(C(C)(C)C)c2)CC(C)(C)N1]

First, compound 1-A is synthesized according to Reaction Equation 1.

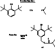

Next, compound 1-A synthesized in Reaction Equation 1 is reacted according to Reaction Equation 2 to synthesize the compound represented by Chemical Formula 1-1 according to an exemplary embodiment of the present invention.

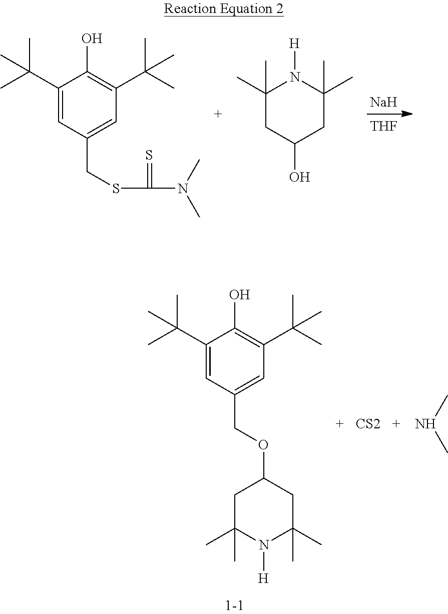

Hereinafter, as an example of the compound represented by Chemical Formula 1, a synthesis of a compound represented by the following Chemical Formula 1-15 will be described in more detail.

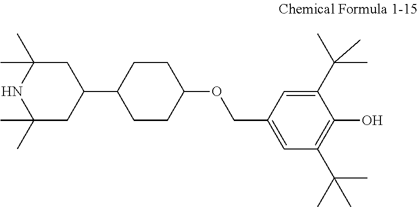 [SMILES: CC1(C)CC(C2CCC(OCc3cc(C(C)(C)C)c(O)c(C(C)(C)C)c3)CC2)CC(C)(C)N1]

First, compound 1-15A is synthesized according to the following Reaction Equation 3, and compound 1-15B is synthesized according to the following Reaction Equation 4.

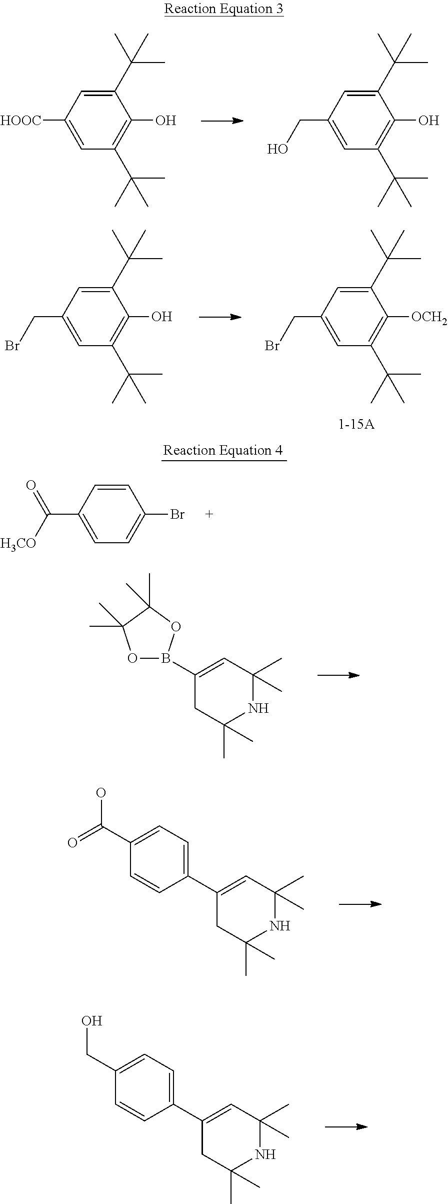

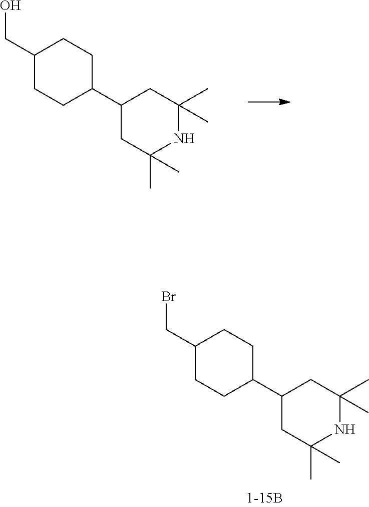 [SMILES: CC1(C)CC(C2CCC(CO)CC2)CC(C)(C)N1 ; CC1(C)CC(C2CCC(CBr)CC2)CC(C)(C)N1]

Synthesized compound 1-15A and compound 1-15B may be synthesized according to the following Reaction Equation 5 to synthesize a compound represented by Chemical Formula 1-15 according to an exemplary embodiment of the present invention.

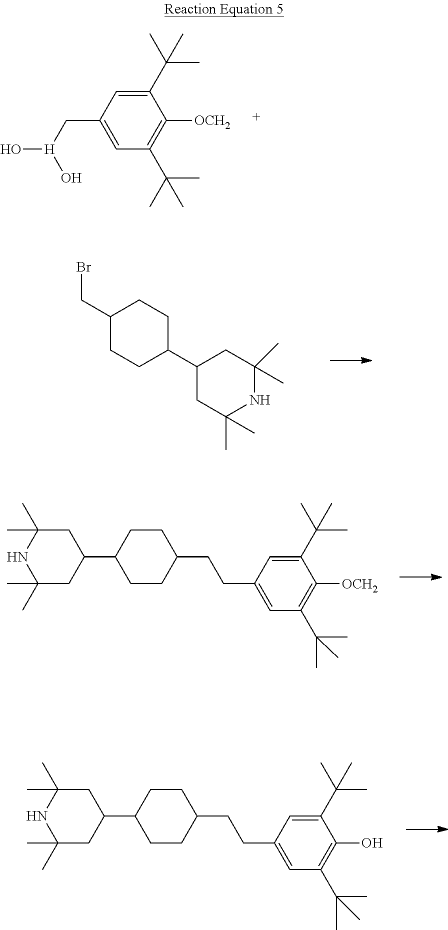

Hereinafter, as an example of the compound represented by Chemical Formula 2, a synthesis of a compound represented by the following Chemical Formula 2-1 will be described in more detail.

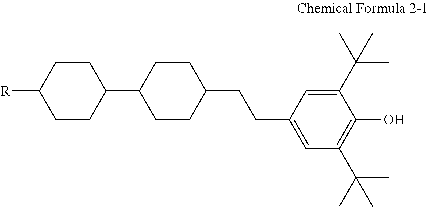

First, compound 2-1A is synthesized according to the following Reaction Equation 6, and compound 2-1B is synthesized according to the following Reaction Equation 7.

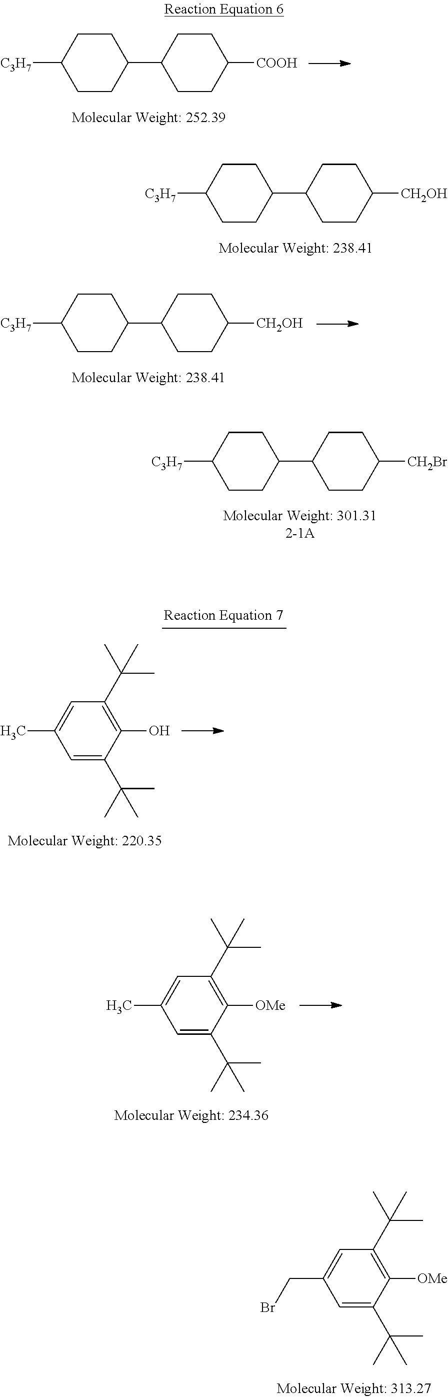

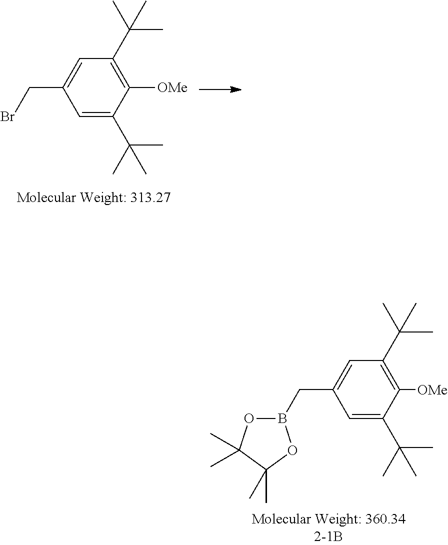 [SMILES: COc1c(C(C)(C)C)cc(CBr)cc1C(C)(C)C ; COc1c(C(C)(C)C)cc(CB2OC(C)(C)C(C)(C)O2)cc1C(C)(C)C]

Next, compounds 2-1A and 2-1B synthesized through Reaction Equations 6 and 7 are synthesized according to the following Reaction Equation 8 to synthesize the compound represented by Chemical Formula 2-1.

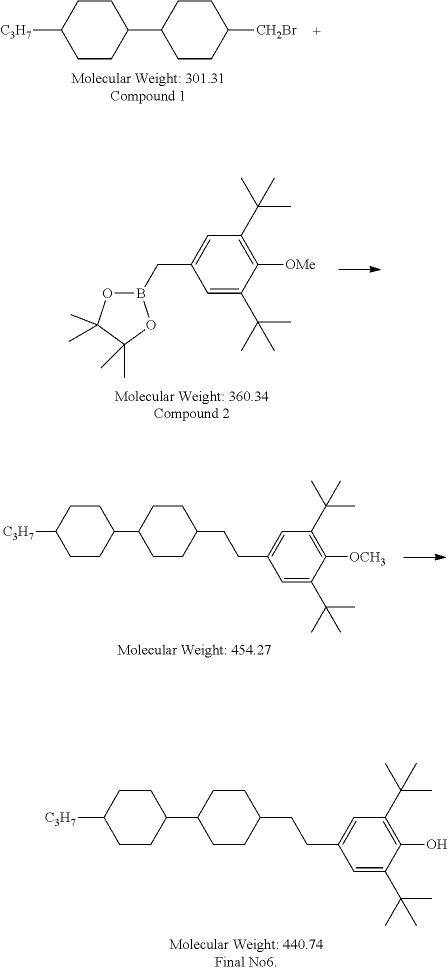

The Reaction Equations or synthesis methods described above may be configured to synthesize the stabilizer according to an exemplary embodiment of the present invention, but exemplary embodiments of the present invention are not limited thereto, and any desired method for synthesizing the compounds represented by Chemical Formulae 1 and 2 may be performed.

The liquid crystal composition according to an exemplary embodiment of the present invention may include the liquid crystal stabilizer and a first class of compounds including the liquid crystal molecules including an alkenyl group. The first class of compounds may include at least one of the compounds represented by the following Chemical Formula 3. However, the first class of compounds is not limited to the exemplary embodiments of the present invention described herein, and the first class of compounds may include any liquid crystal molecule including the alkenyl group at an end group of the liquid crystal molecule.

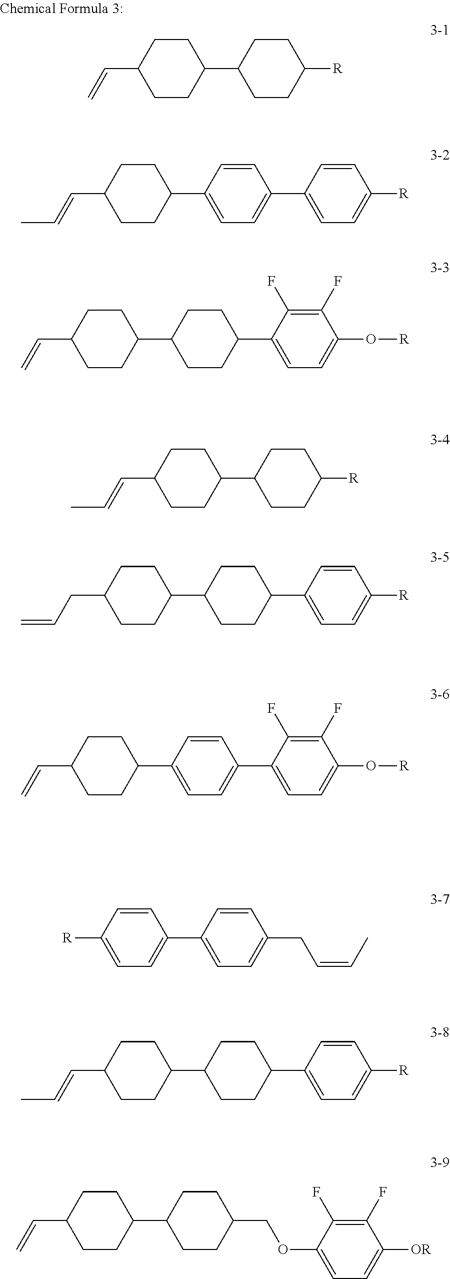

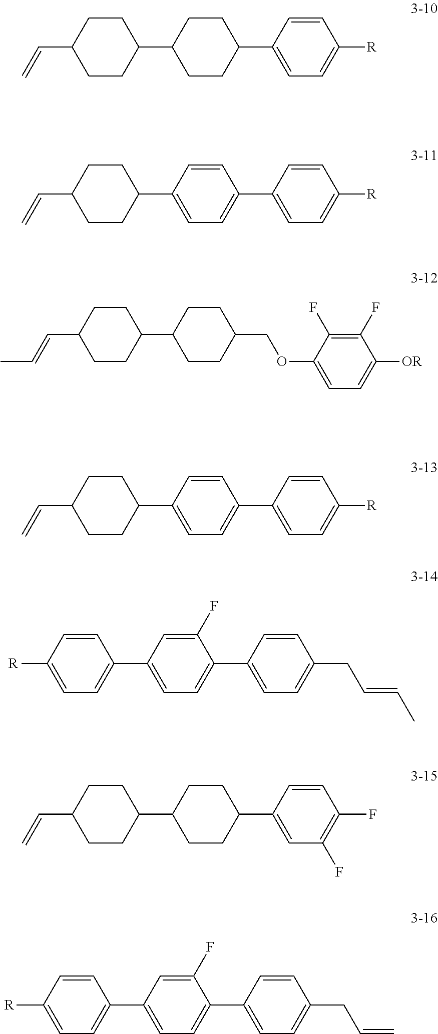

R may be an alkyl group or an alkoxy group having 1 to 5 carbon atoms.

The liquid crystal composition according to an exemplary embodiment of the present invention may include the liquid crystal stabilizer. The liquid crystal composition may include the liquid crystal stabilizer in a content of from about 0.0001 wt % to about 10 wt % based on the entire liquid crystal composition. The liquid crystal composition including the liquid crystal stabilizer may reduce or prevent the occurrence of a reaction between the liquid crystal molecules and radicals without vitiating physical properties of the liquid crystal composition.

In the liquid crystal composition according to an exemplary embodiment of the present invention, alkenyl-based liquid crystal molecules having a relatively low viscosity may be included to increase a response speed. Byproducts generated while an electric field exposure process and a fluorescent exposure process are performed may be reduced or prevented by the liquid crystal stabilizer according to an exemplary embodiment of the present invention. Reliability of the display device may be increased by reducing the linear afterimages and the stains by using the liquid crystal composition including the liquid crystal stabilizer according to an exemplary embodiment of the present invention.

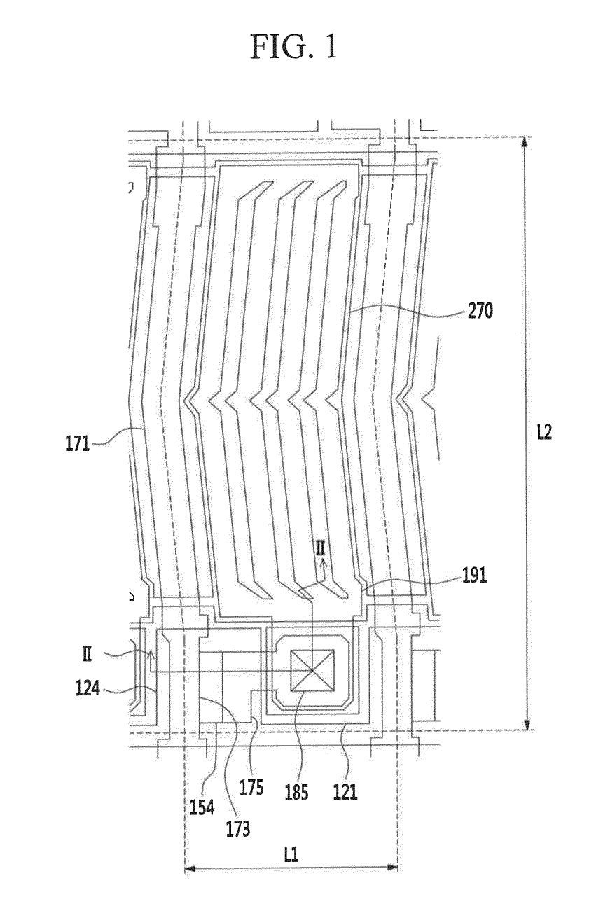

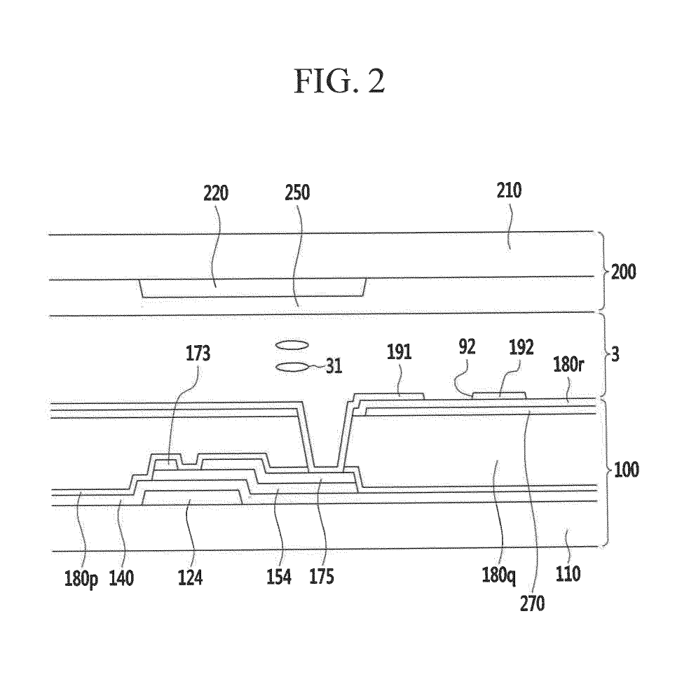

Hereinafter, a liquid crystal display including the liquid crystal stabilizer according to an exemplary embodiment of the present invention will be described with reference to FIGS. 1 to 2. FIG. 1 is a top plan view of a pixel according to an exemplary embodiment of the present invention. FIG. 2 is a cross-sectional view taken along line II-II of FIG. 1.

In a thin film transistor array panel of the liquid crystal display according to an exemplary embodiment of the present invention, a gate line 121 including a gate electrode 124 may be disposed on a first insulation substrate 110, which may include a transparent material. The transparent material may include glass or a plastic.

A gate conductor may include an aluminum-based metal such as aluminum (Al) or an aluminum alloy, a silver-based metal such as silver (Ag) or an silver alloy, a copper-based metal such as copper (Cu) or a copper alloy, a molybdenum-based metal such as molybdenum (Mo) or a molybdenum alloy, chromium (Cr), tantalum (Ta), and/or titanium (Ti).

A gate insulation layer 140 including silicon nitride (SiNx) or silicon oxide (SiOx) may be disposed on the gate conductor. The gate insulation layer 140 may have a multilayered structure including at least two insulation layers having different physical properties from each other.

A semiconductor layer 154 may be disposed on the gate insulation layer 140. A data line 171 including a source electrode 173 and a drain electrode 175 may be disposed on the semiconductor layer 154 and the gate insulation layer 140.

The data line 171 may include a wide end portion configured to connect with another layer or an external driving circuit. The data line 171 may transmit a data signal. The data line 171 may extend in a vertical direction and may cross the gate line 121.

The data line 171 may have a first bent portion having a bent shape. The bent portion of the data line 171 may increase transmittance of the liquid crystal display. Bent portions of the data line 171 may contact each other in an intermediate region of a pixel region to form a V shape. A second bent portion that is bent to form a predetermined angle with the first bent portion may be disposed in the intermediate region of the pixel region.

The source electrode 173 may be a portion of the data line 171, and may be disposed on the same line as the data line 171. The drain electrode 175 may extend in parallel with the source electrode 173. Accordingly, the drain electrode 175 may be parallel with a portion of the data line 171.

The gate electrode 124, the source electrode 173, and the drain electrode 175 may form one thin film transistor (TFT) together with the semiconductor layer 154. A channel of the TFT may be disposed in the semiconductor layer 154 between the source electrode 173 and the drain electrode 175.

The liquid crystal display according to an exemplary embodiment of the present invention may include the source electrode 173 disposed on the same line as the data line 171 and the drain electrode 175 extending in parallel to the data line 171. The source electrode 173 may increase a width of the TFT without increasing an area occupied by a data conductor, and thus an opening ratio of the liquid crystal display may be increased.

The data conductor including the data line 171 may include a refractory metal such as molybdenum, chromium, tantalum, and titanium, or an alloy thereof. The data conductor may have a multilayered structure including a refractory metal layer (not illustrated) and a low resistance conductive layer (not illustrated).

The multilayered structure may include a double layer. The double layer may include a chromium or molybdenum (alloy) lower layer and an aluminum (alloy) upper layer. The multilayered structure may include a triple layer. The triple layer may include a molybdenum (alloy) lower layer, an aluminum (alloy) intermediate layer, and a molybdenum (alloy) upper layer. However, the data line 171 and the drain electrode 175 may include various metals, and exemplary embodiments of the present invention are not limited to particular metals.

A first passivation layer 180p may be disposed on exposed portions of the data conductors 171, 173, and 175, the gate insulation layer 140, and the semiconductor layer 154. The first passivation layer 180p may include an organic insulation material or an inorganic insulation material.

A second passivation layer 180q may be disposed on the first passivation layer 180p. The second passivation layer 180q may be omitted.

The second passivation layer 180q may be a color filter. When the second passivation layer 180q is the color filter, the second passivation layer 180q may intrinsically display any one of the primary colors. Examples of the primary colors may include primary colors such as red, green, blue, yellow, cyan and magenta. The color filter may display a mixed color of the primary colors or white in addition to the primary colors.

A common electrode 270 may be disposed on the second passivation layer 180q. The common electrode 270 may have a shape corresponding to the surface of the second passivation layer 180q. The common electrode 270 may be disposed on an entire surface of the substrate 110 and may have a whole plate shape. The common electrode 270 may have an opening disposed in a region of the drain electrode 175. The common electrode 270 may have a flat plate shape.

The common electrodes 270 may be disposed in adjacent pixels, which may be connected to each other to receive a common voltage having a predetermined size supplied from the outside of a display region.

A third passivation layer 180r may be disposed on the common electrode 270. The third passivation layer 180r may include an organic insulation material or an inorganic insulation material.

A pixel electrode 191 may be disposed on the third passivation layer 180r. The pixel electrode 191 may include a curved edge that is substantially parallel with the first bent portion and the second bent portion of the data line 171. The pixel electrode 191 may have a plurality of cutouts, and may include a plurality of slits defined by the plurality of cutouts.

A contact hole 185 through which the drain electrode 175 is exposed may be disposed in the first passivation layer 180p, the second passivation layer 180q, and the third passivation layer 180r. The pixel electrode 191 may be physically and electrically connected to the drain electrode 175 through the contact hole 185 to receive a voltage from the drain electrode 175.

An alignment layer may be disposed on the pixel electrode 191 and the third passivation layer 180r. The alignment layer may be a horizontal alignment layer and may be rubbed in a predetermined direction.

An upper display panel 200 will be described below in more detail.

A light blocking member 220 may be disposed on a second insulation substrate 210. A flat layer 250 may be disposed on the light blocking member 220.

In an exemplary embodiment of the present invention, the color filter may be disposed on the lower display panel and the light blocking member 220 may be disposed on the upper display panel, but exemplary embodiments of the present invention are not limited thereto. Both the color filter and the light blocking member 220 may be disposed on a lower display panel 100. Both the color filter and the light blocking member 220 may be disposed on the upper display panel 200. Positions of the color filter and the light blocking member 220 may be changed, as desired.

A liquid crystal layer 3 according to an exemplary embodiment of the present invention may have a positive dielectric anisotropy. The liquid crystal layer 3 may include a liquid crystal composition having the positive dielectric anisotropy. Long axes of liquid crystal molecules 31 of the liquid crystal layer 3 may be horizontally aligned with respect to surfaces of the lower and upper display panels 100 and 200 when an electric field is not applied to the liquid crystal layer 3.

The liquid crystal display according to an exemplary embodiment of the present invention may include the liquid crystal stabilizer according to exemplary embodiments of the present invention. The liquid crystal stabilizer according to an exemplary embodiment of the present invention may include at least one of the compounds represented by the following Chemical Formulae 1 and 2.

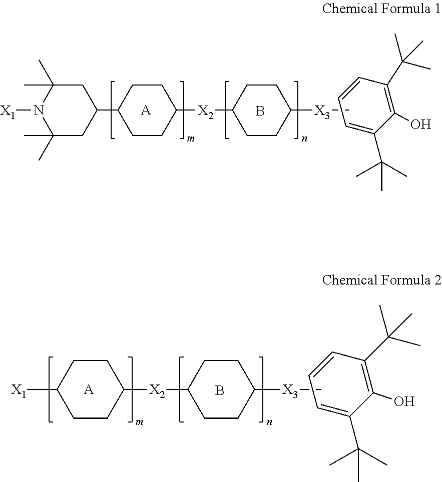

X₁ may be any one of H, O, OR, and R. X₂ may be any one of O, OCO, OR, RO, NOR, and R. X₃ may be any one of O, OCO, OR, RO, NOR, and R. A and B may each independently include one or more of a cyclohexyl group, a cyclic ether group, and a phenyl group. R may be an alkyl group having 1 to 5 carbon atoms, and m and n may each independently be a natural number from 0 to 2.

According to an exemplary embodiment of the present invention, the pixel electrode 191 may be disposed on the common electrode 270, but exemplary embodiments of the present invention are not limited thereto, and the common electrode 270 may be disposed on the pixel electrode 191. According to an exemplary embodiment of the present invention, the pixel electrode 191 and the common electrode 270 may be disposed on the same insulation substrate (e.g., the first insulation substrate 110 or the second insulation substrate 210), but exemplary embodiments of the present invention are not limited thereto, and the pixel electrode 191 may be disposed on the first insulation substrate 110 and the common electrode 270 may be disposed on the second insulation substrate 210.

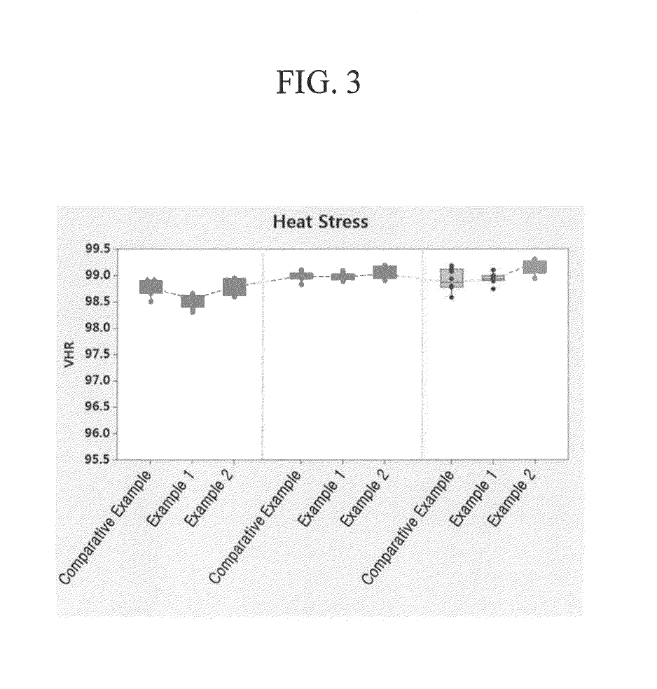

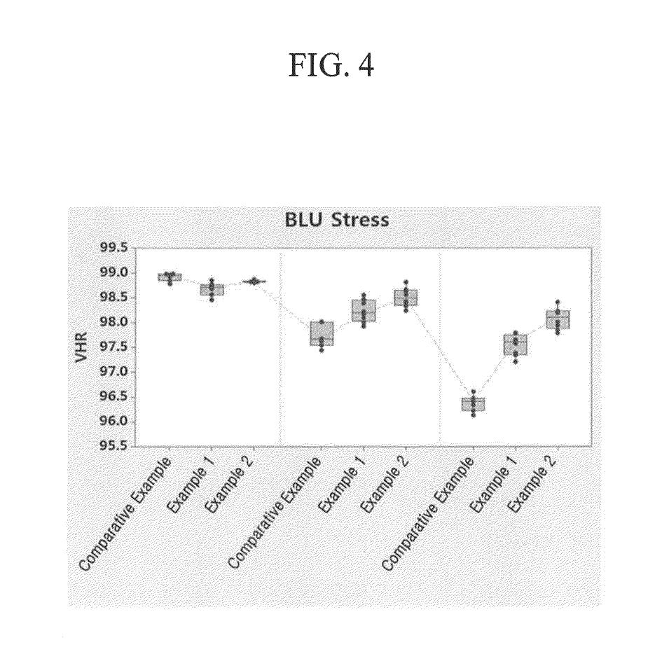

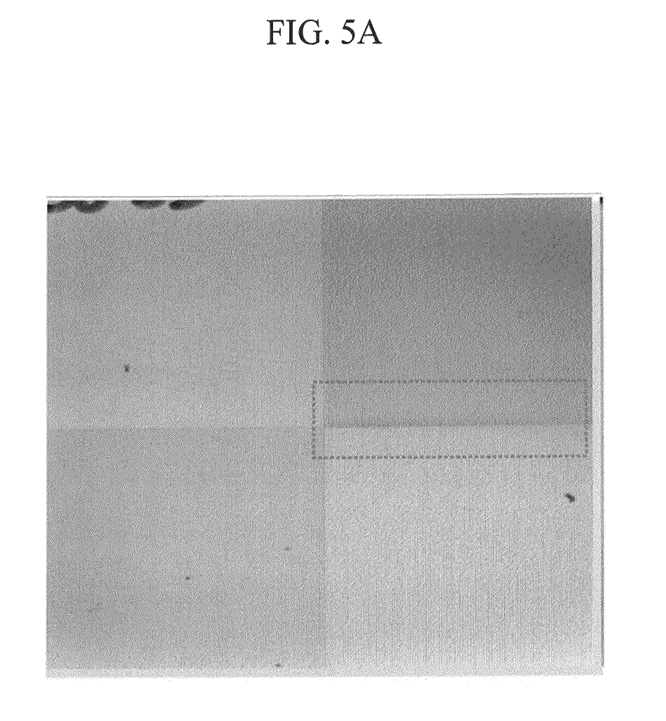

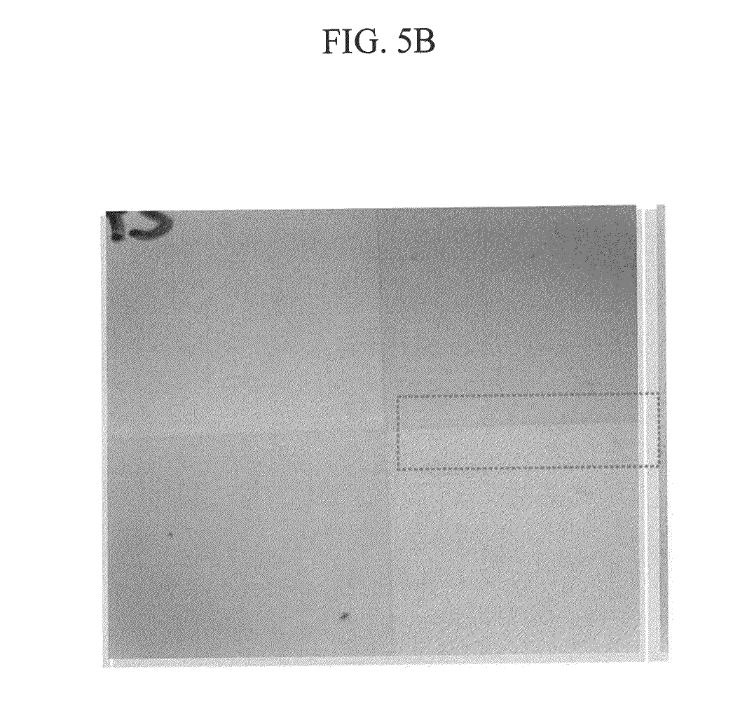

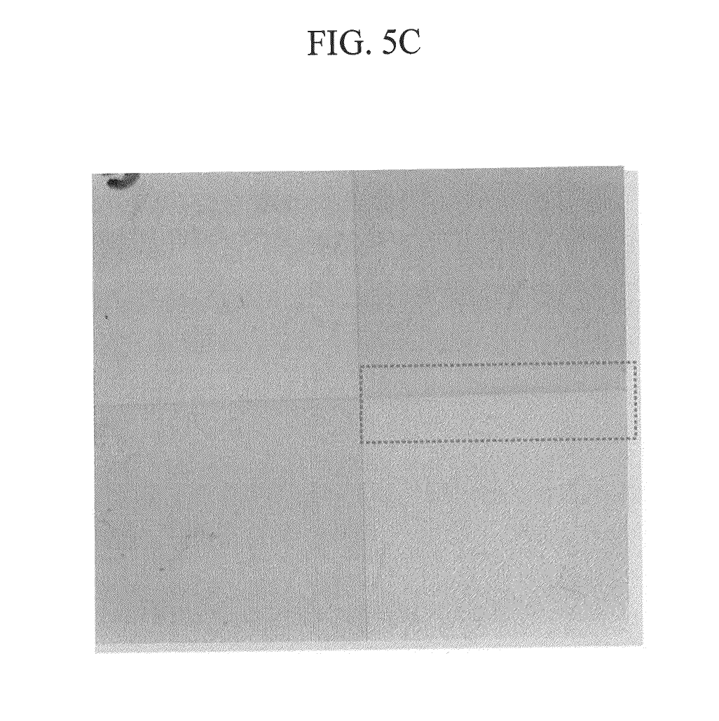

Hereinafter, exemplary embodiments of the present invention and Comparative Examples will be described with reference to FIGS. 3, 4, 5A, 5B and 5C. FIGS. 3 and 4 are voltage holding ratio graphs according to exemplary embodiments of the present invention and the Comparative Examples. FIGS. 5A to 5C are images showing whether there is an afterimage or not in exemplary embodiments of the present invention and the Comparative Examples.

Examples 1 and 2 according to exemplary embodiments of the present invention and the Comparative Example may include at least one of the following liquid crystal molecules, respectively. Liquid crystal molecules 1 to 9 of Example 1 may include the same content as liquid crystal molecules 1 to 9 of Example 2. Example 1 may include a liquid crystal stabilizer (e.g., compound 10 illustrated below and Example 1-1 described above according to exemplary embodiments of the present invention) in a content of 300 ppm. Example 2 may include the liquid crystal stabilizer (e.g., Example 1-15 described above according to exemplary embodiments of the present invention) in a content of 500 ppm. The Comparative Example does not include the liquid crystal stabilizer according to exemplary embodiments of the present invention. R of the following Chemical Formulae may be an alkyl group or an alkoxy group having 1 to 5 carbon atoms.

### Example 1

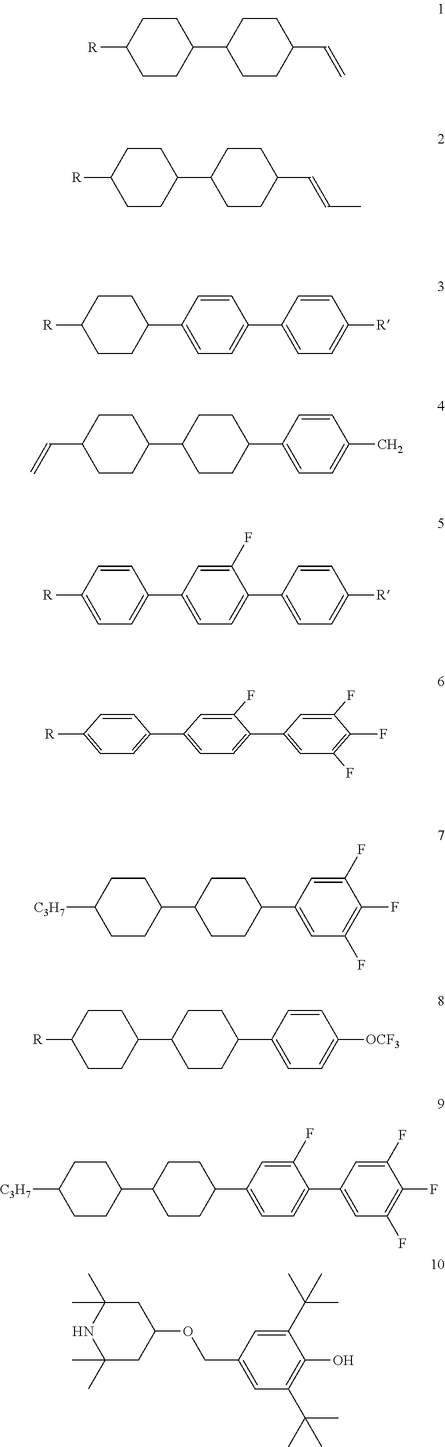

### Example 2

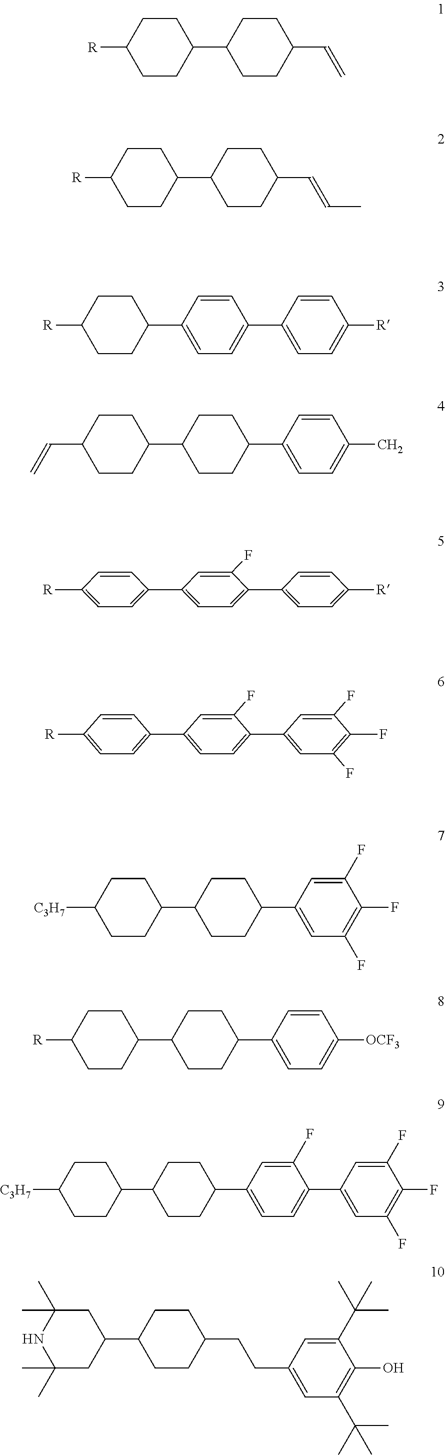

### Comparative Example

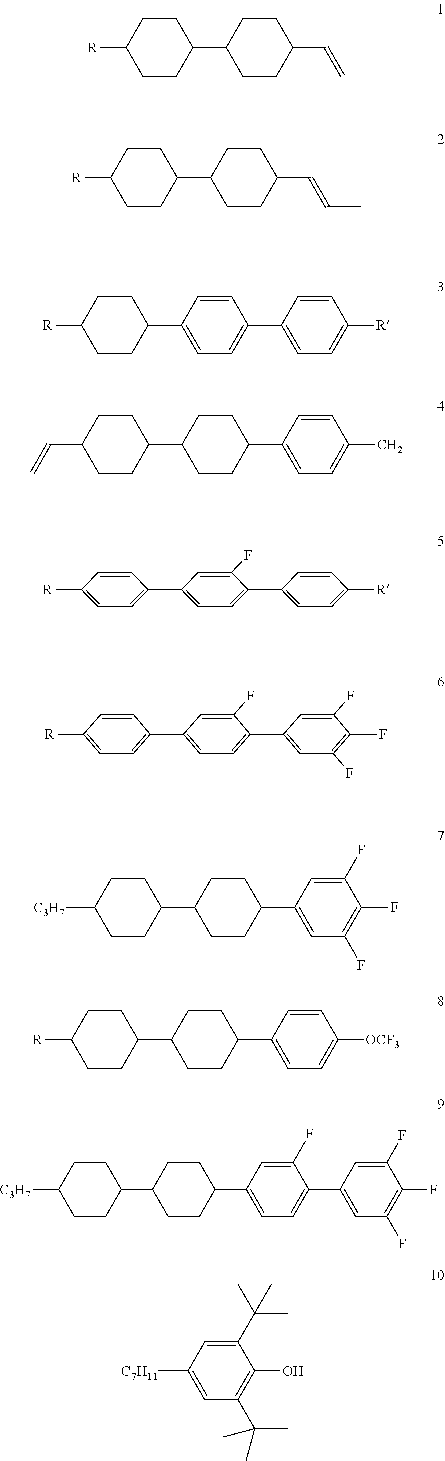

FIG. 3 illustrates voltage holding ratio (VHR) over time while heat is applied to the display panel. Referring to FIG. 3, in Examples 1 and 2 according to exemplary embodiments of the present invention, as time passes, the VHR might not be reduced, but rather may be increased. In the Comparative Example, as time passes (e.g., 168 hours to 336 hours) the VHR may be reduced.

FIG. 4 illustrates voltage holding ratio (VHR) over time while light is irradiated on the display panel. Referring to FIG. 4, in Examples 1 and 2 according to exemplary embodiments of the present invention, the VHR may be partially reduced. For example, the VHR may be reduced from about 99 to about 97. In the Comparative Example, the VHR may be reduced from 99 to about 96 as time passes.

According to exemplary embodiments of the present invention, the reaction of the liquid crystal molecules by exposure to heat or light may be relatively unlikely to occur, and thus the VHR may be substantially maintained.

FIG. 5A illustrates an afterimage for the Comparative Example. FIG. 5B illustrates an afterimage for Example 1. FIG. 5C illustrates an afterimage for Example 2. In FIGS. 5A to 5C, afterimage patterns maintained over a relatively long period of time in four regions are illustrated.

Referring to FIG. 5A, a linear afterimage is illustrated between upper and lower regions at the right side of FIG. 5A. Referring to FIGS. 5B and 5C, a relatively faint linear afterimage is illustrated between the upper and lower regions at the right side of FIGS. 5B and 5C. Comparing FIG. 5A to FIGS. 5B and 5C, it is illustrated that the linear afterimage was significantly reduced in Examples 1 and 2 according to exemplary embodiments of the present invention compared with the Comparative Example.

The liquid crystal stabilizer according to an exemplary embodiment of the present invention may be included in the liquid crystal composition and the liquid crystal display to reduce or prevent an occurrence of the linear afterimage and to reduce or prevent a generation of impurities in the liquid crystal composition and the liquid crystal display.

While the present invention has been shown and described with reference to the exemplary embodiments thereof, it will be apparent to those of ordinary skill in the art that various changes in form and detail may be made thereto without departing from the spirit and scope of the present invention.

What is claimed is:

1. A liquid crystal stabilizer comprising:
at least one of the compounds represented by the following Chemical Formulae 1 and 2: 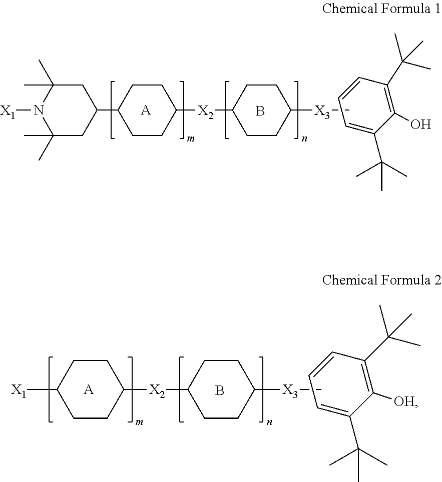
wherein X₁ is any one of H, O, OR, and R, X₂ is any one of O, OCO, OR, RO, NOR, and R, X₃ is any one of O, OCO, OR, RO, NOR, and R, A and B each independently include one or more of a cyclohexyl group, a cyclic ether group, and a phenyl group, R is an alkyl group having 1 to 5 carbon atoms, and m and n are each independently a natural number from 0 to 2.

2. The liquid crystal stabilizer of claim 1, wherein the compound represented by Chemical Formula 1 is at least one of the compounds represented by the following Chemical Formulae 1-1 to 1-28: 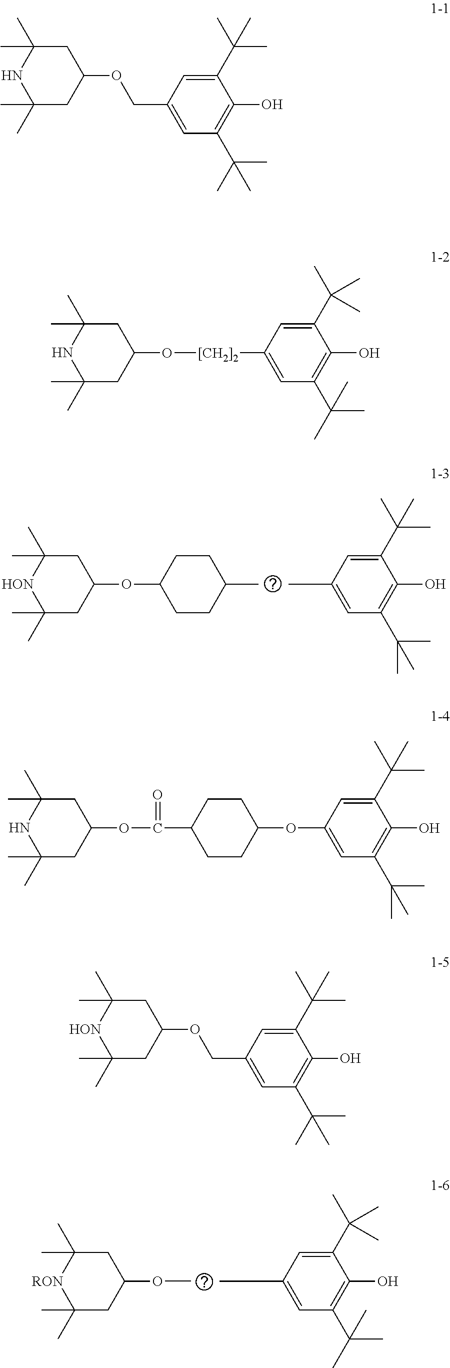 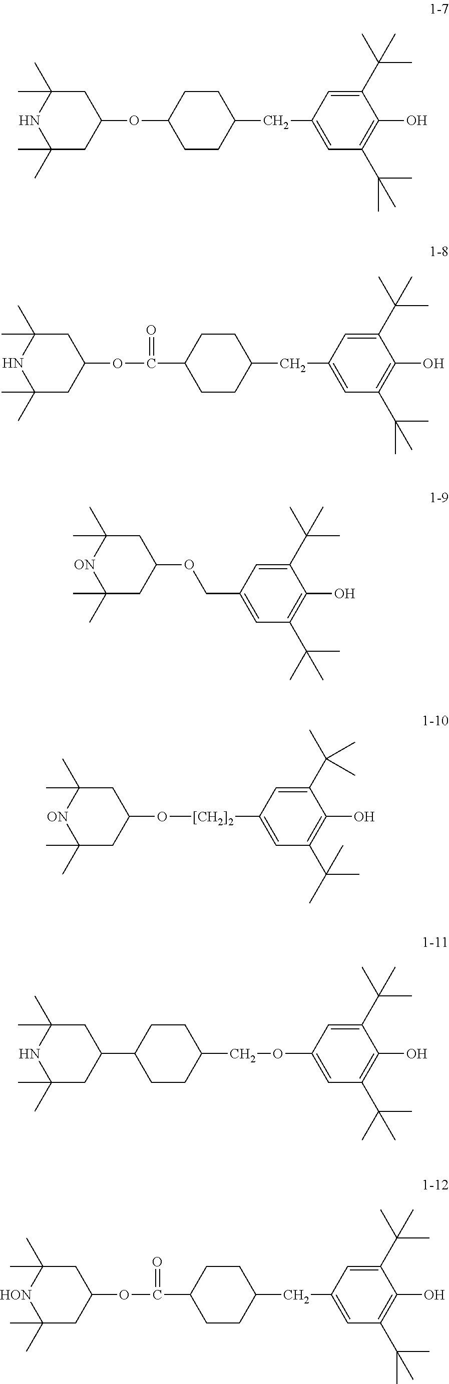 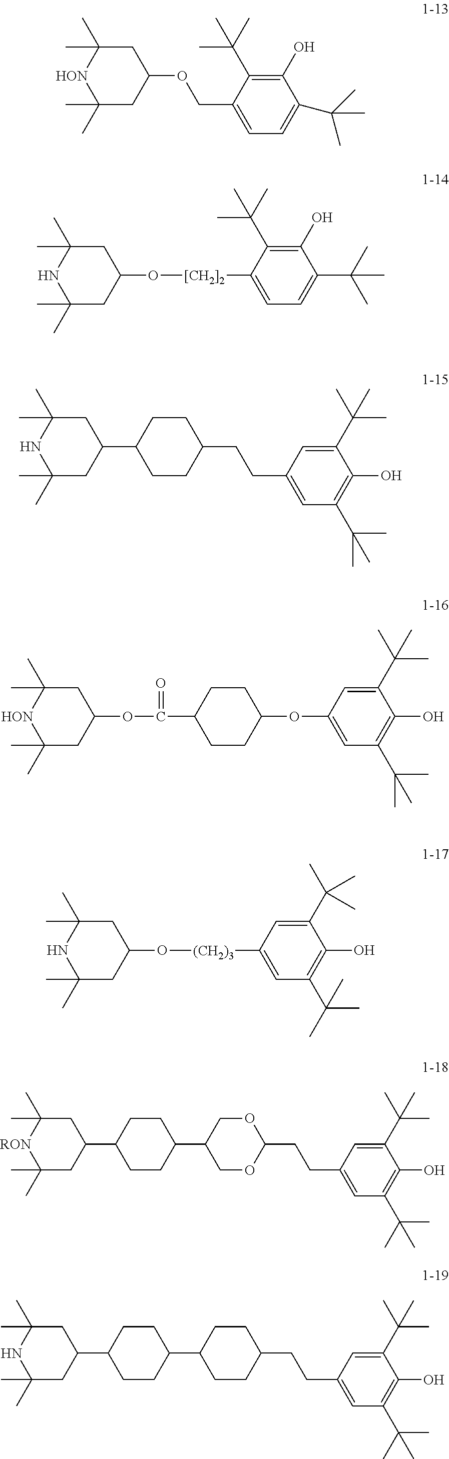 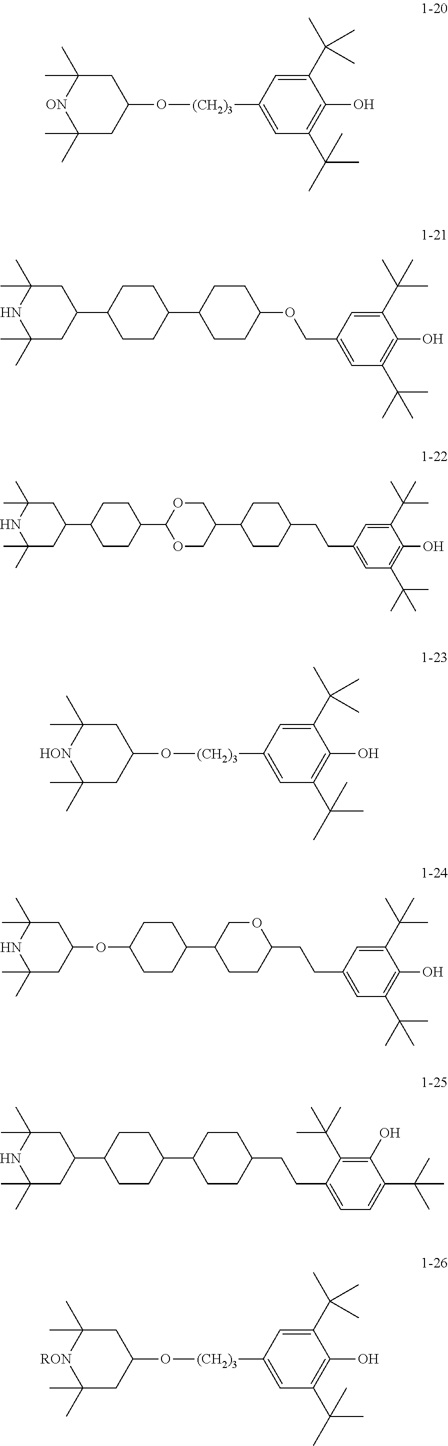 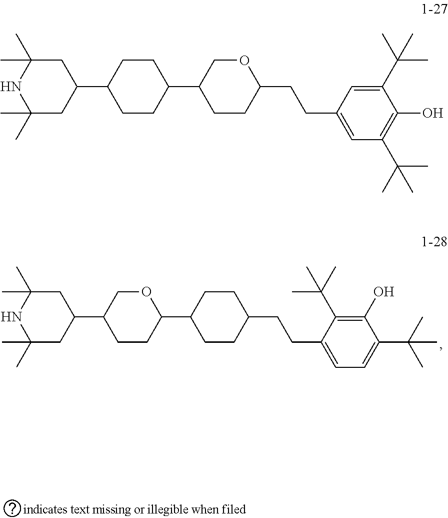
wherein R is an alkyl group having 1 to 5 carbon atoms.

3. The liquid crystal stabilizer of claim 1, wherein the compound represented by Chemical Formula 2 is at least one of the compounds represented by the following Chemical Formulae 2-1 to 2-16: 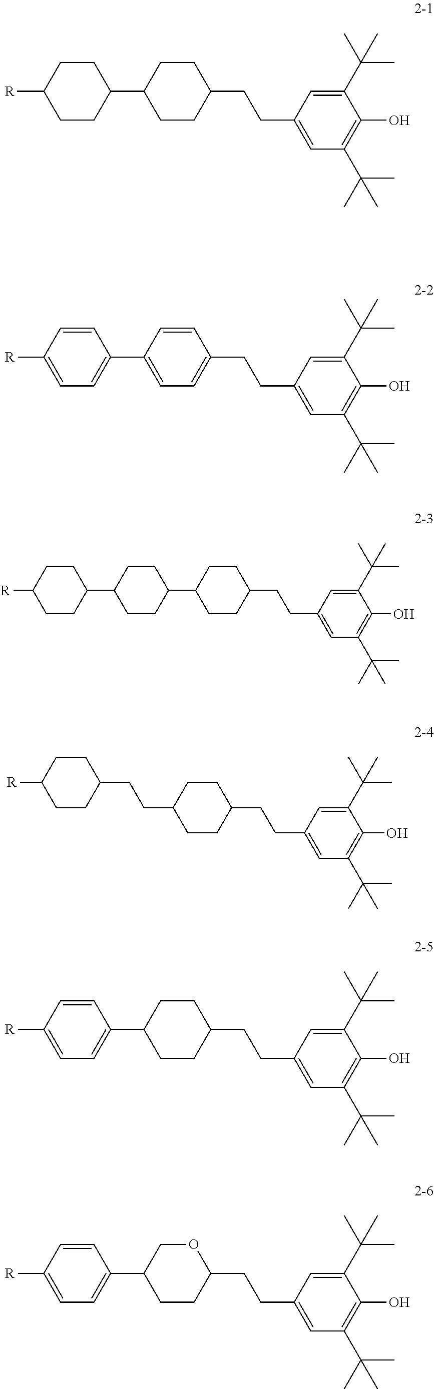 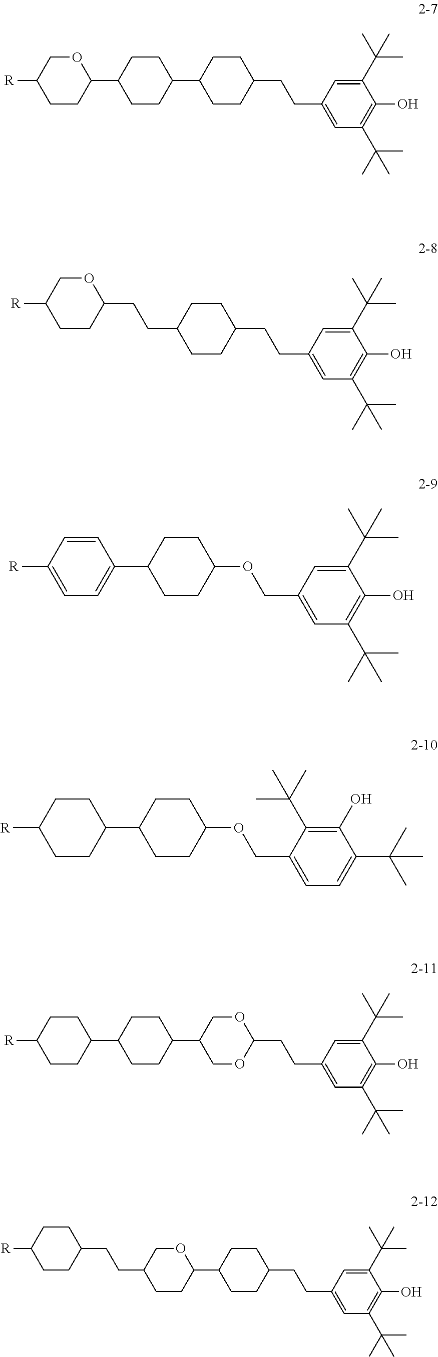 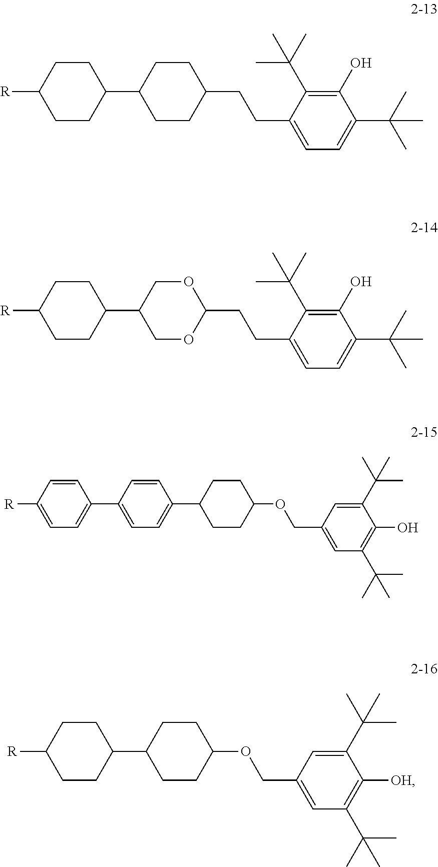
wherein R is an alkyl group having 1 to 5 carbon atoms.

4. A liquid crystal composition comprising:
a first class of compounds including an alkenyl group; and
a liquid crystal stabilizer including at least one of the compounds represented by the following Chemical Formulae 1 and 2: 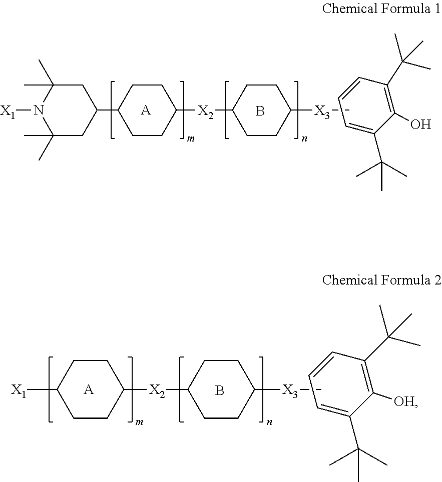
wherein X₁ is any one of H, O, OR, and R, X₂ is any one of O, OCO, OR, RO, NOR, and R, X₃ is any one of O, OCO, OR, RO, NOR, and R, A and B each independently include one or more of a cyclohexyl group, a cyclic ether group, and a phenyl group, R is an alkyl group having 1 to 5 carbon atoms, and m and n are each independently a natural number from 0 to 2.

5. The liquid crystal composition of claim 4, wherein the liquid crystal stabilizer is included in the liquid crystal composition in a content of from about 0.0001 wt % to about 10 wt % based on the liquid crystal composition.

6. The liquid crystal composition of claim 4, wherein the compound represented by Chemical Formula 1 is at least one of the compounds represented by the following Chemical Formulae 1-1 to 1-28: 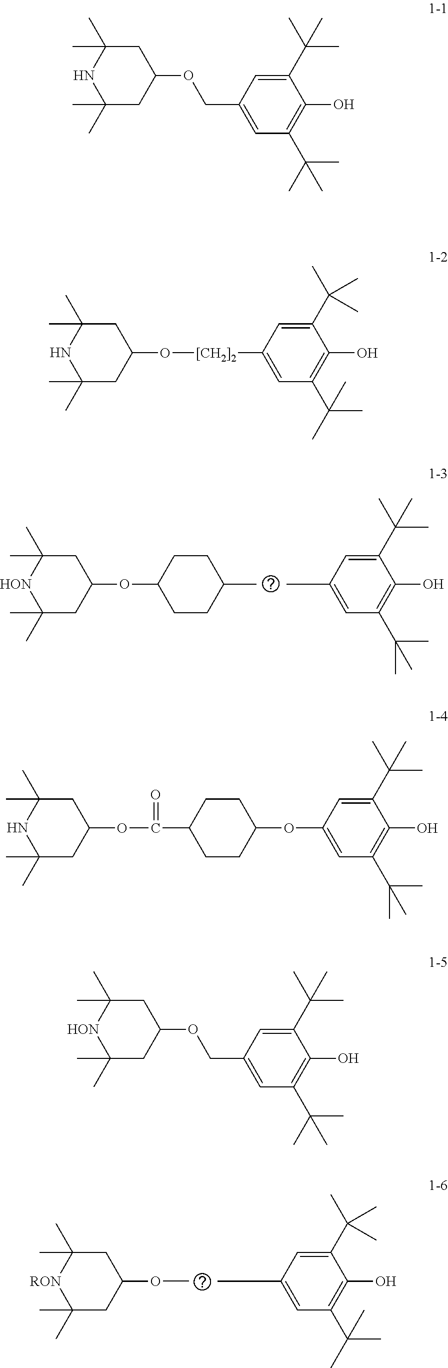 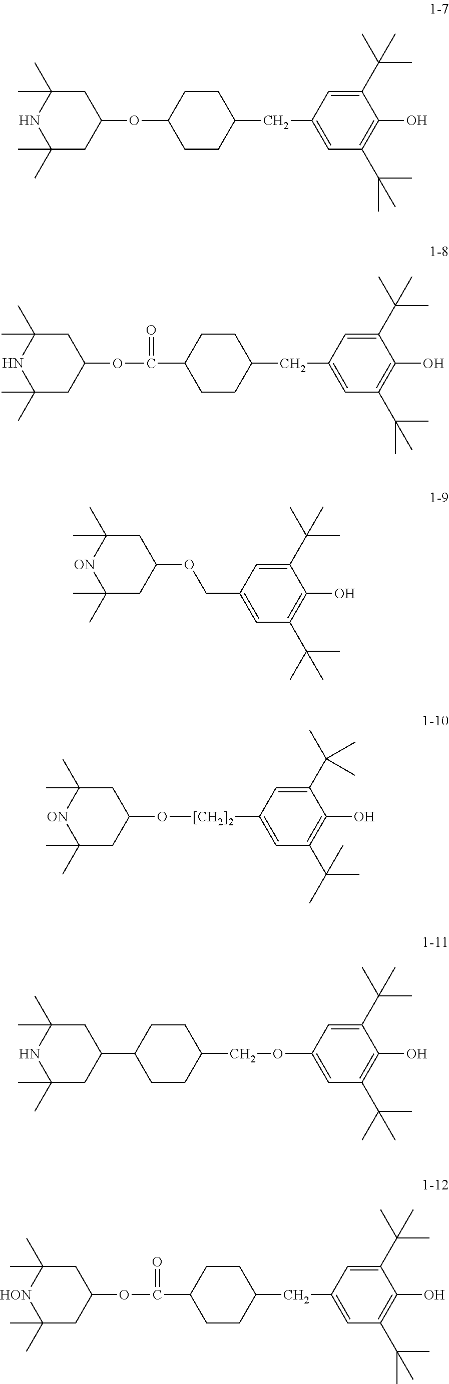 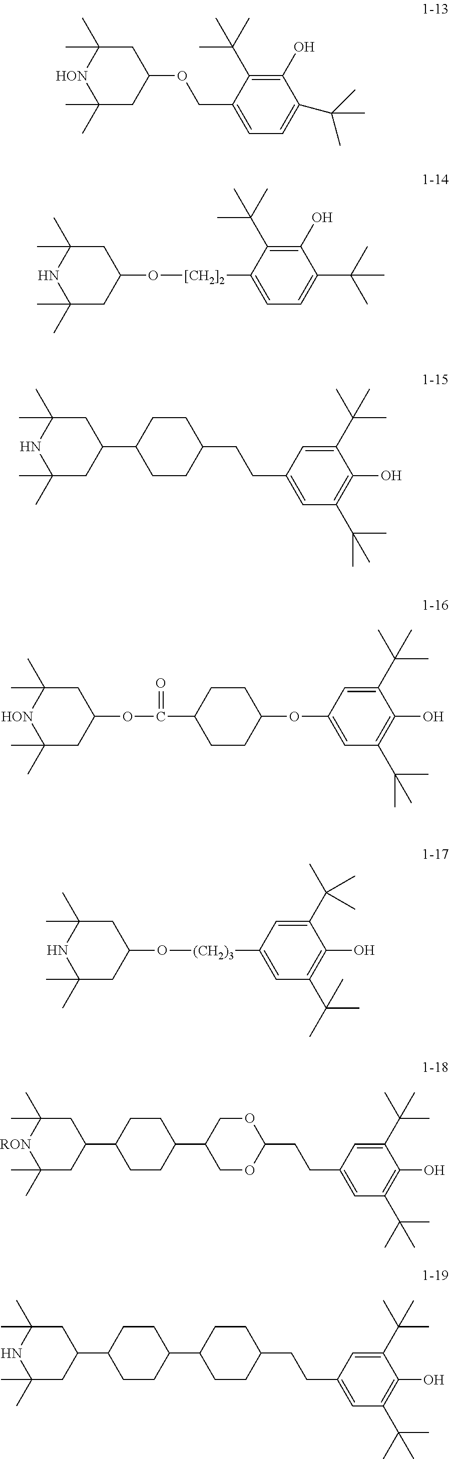 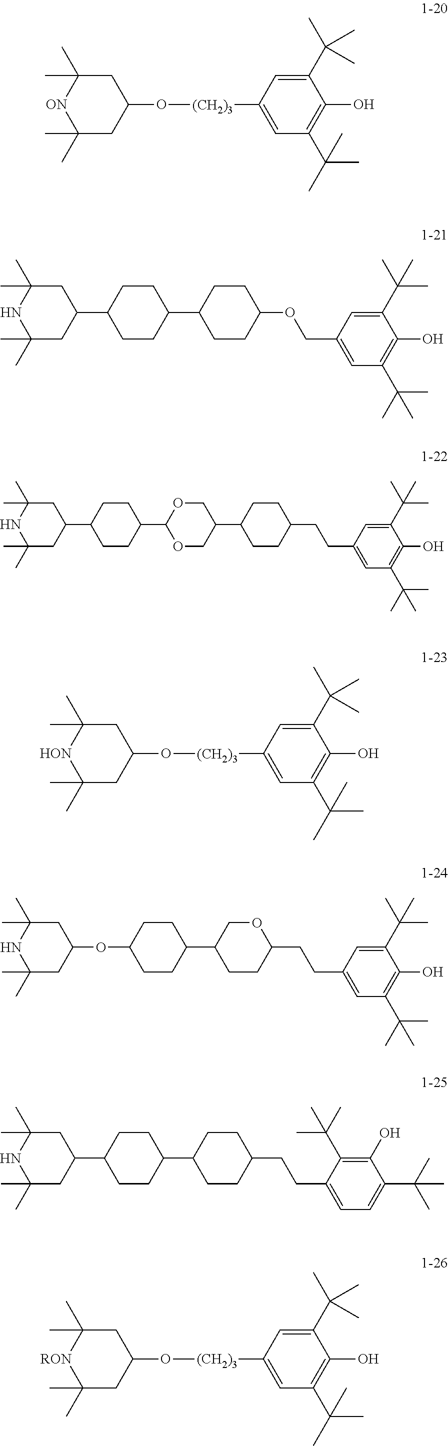 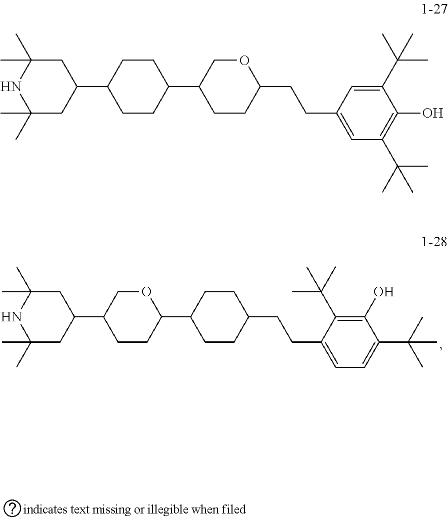
wherein R is an alkyl group having 1 to 5 carbon atoms.

7. The liquid crystal composition of claim 4, wherein the compound represented by Chemical Formula 2 is at least one of the compounds represented by the following Chemical Formulae 2-1 to 2-16: 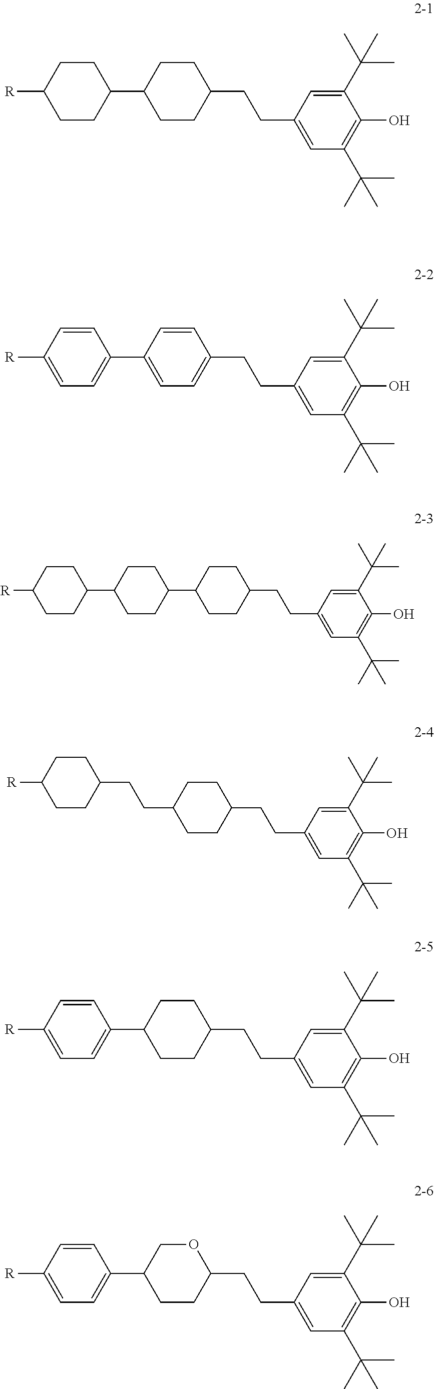 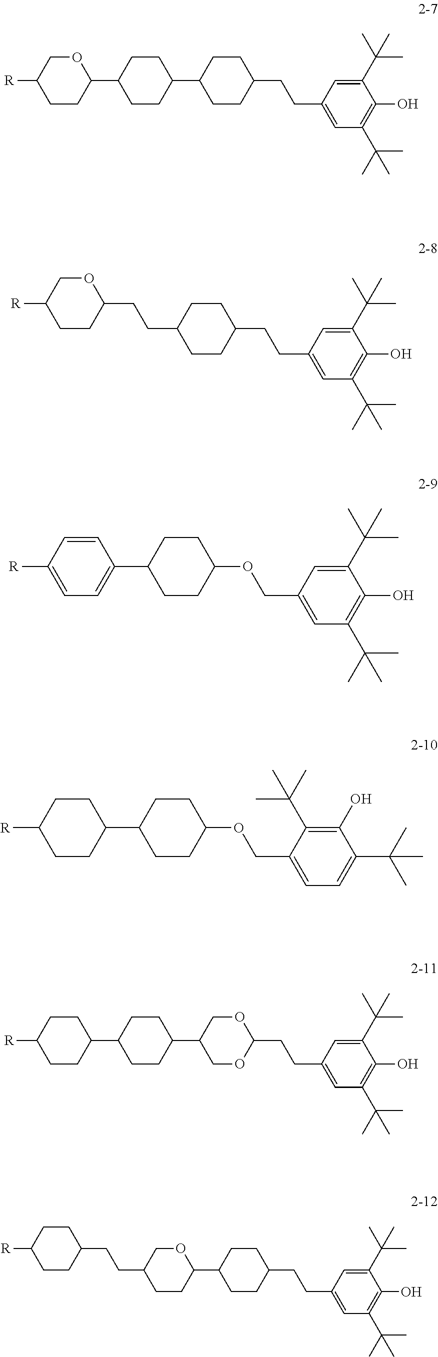 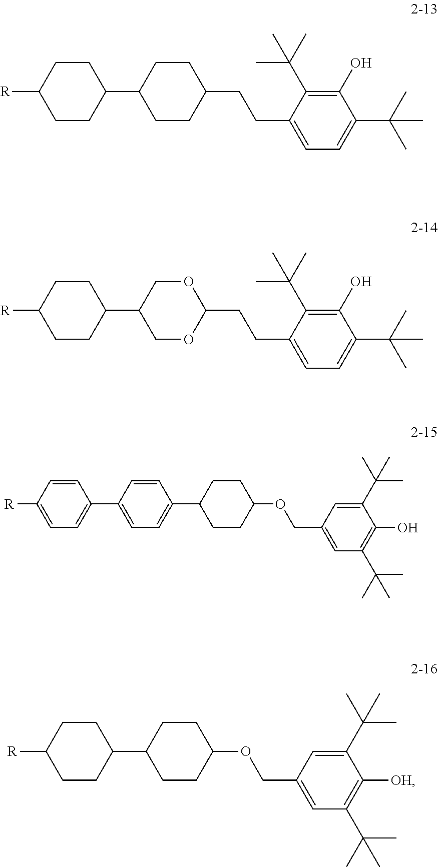
wherein R is an alkyl group having 1 to 5 carbon atoms.

8. The liquid crystal composition of claim 4, wherein the first class of compounds includes at least one of the compounds represented by the following Chemical Formulae 3-1 to 3-16: 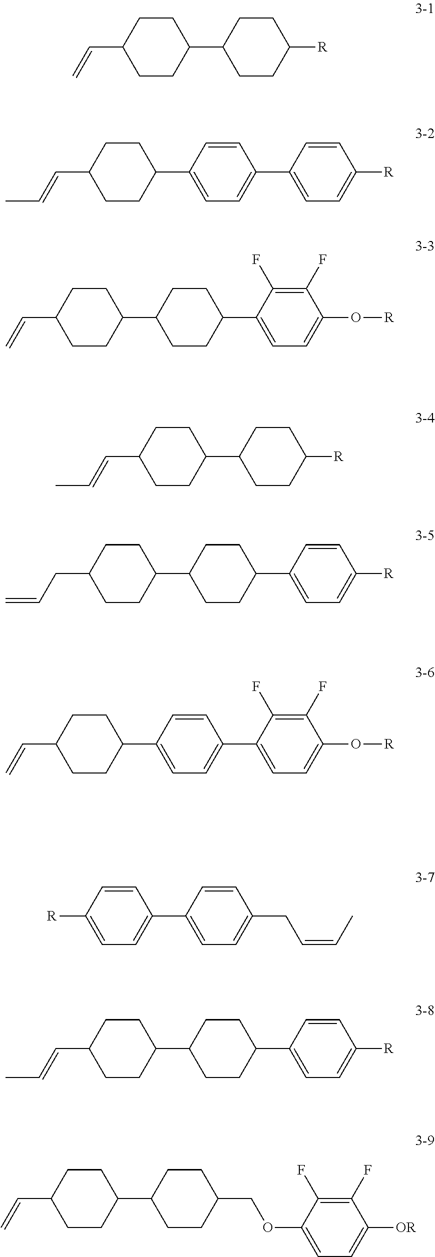 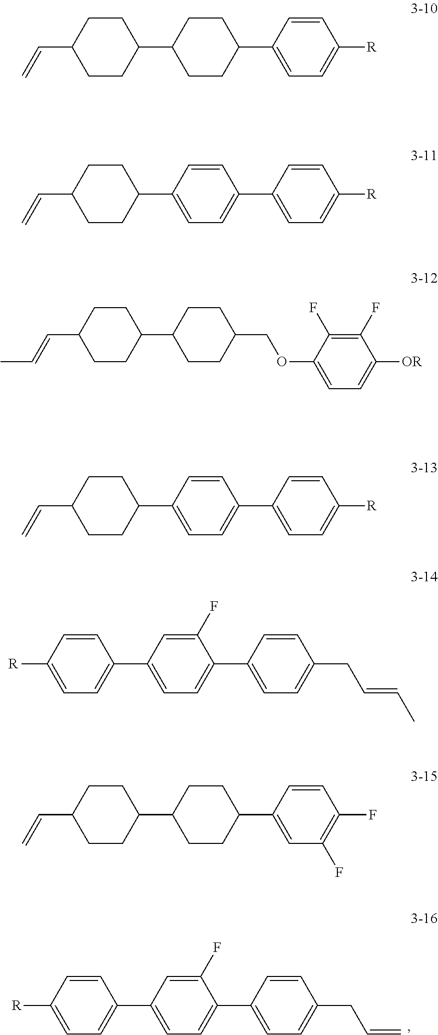
wherein R is an alkyl group or an alkoxy group having 1 to 5 carbon atoms.

9. A liquid crystal display comprising:
a first insulation substrate;
a thin film transistor disposed on the first insulation substrate;
a pixel electrode disposed on the thin film transistor;
a common electrode disposed on the first insulation substrate, wherein the common electrode is insulated from the pixel electrode;
a second insulation substrate facing the first insulation substrate and spaced apart from the first insulation substrate; and
a liquid crystal layer disposed between the first insulation substrate and the second insulation substrate,
wherein the liquid crystal layer includes:
a first class of compounds including an unsaturated bond, and
a liquid crystal stabilizer including at least one of the compounds represented by the following Chemical Formulae 1 and 2: 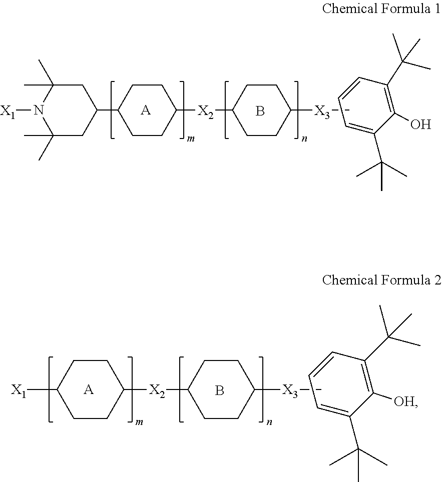
wherein X₁ is any one of H, O, OR, and R, X₂ is any one of O, OCO, OR, RO, NOR, and R, X₃ is any one of O, OCO, OR, RO, NOR, and R, A and B each independently include one or more of a cyclohexyl group, a cyclic ether group, and a phenyl group, R is an alkyl group having 1 to 5 carbon atoms, and m and n are each independently a natural number from 0 to 2.

10. The liquid crystal display of claim 9, wherein the liquid crystal layer includes the liquid crystal stabilizer in a content of from about 0.0001 wt % to about 10 wt % based on the liquid crystal layer.

11. The liquid crystal display of claim 9, wherein the compound represented by Chemical Formula 1 is at least one of the compounds represented by the following Chemical Formulae 1-1 to 1-28: 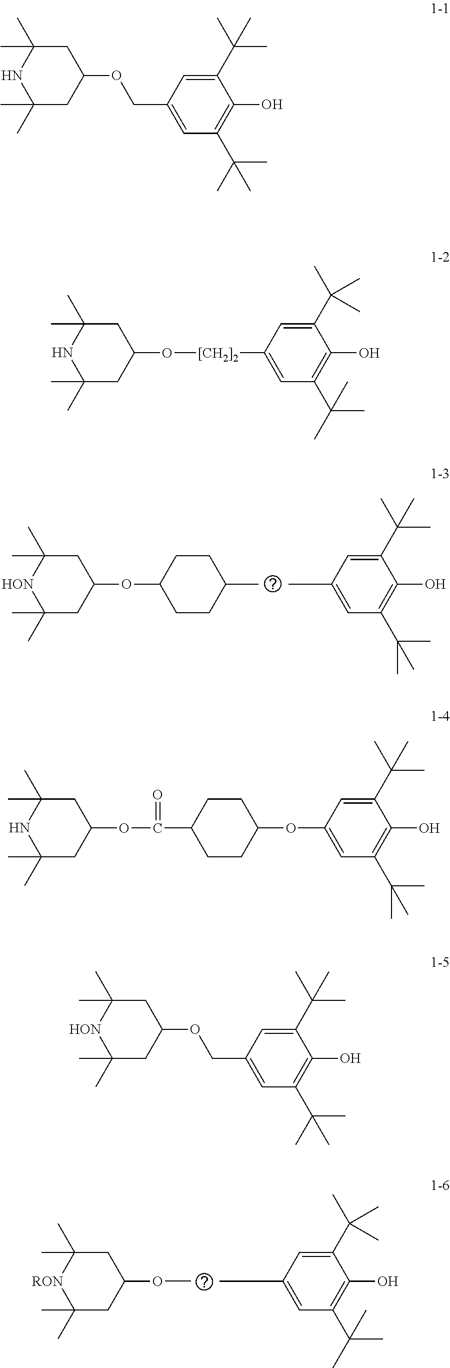 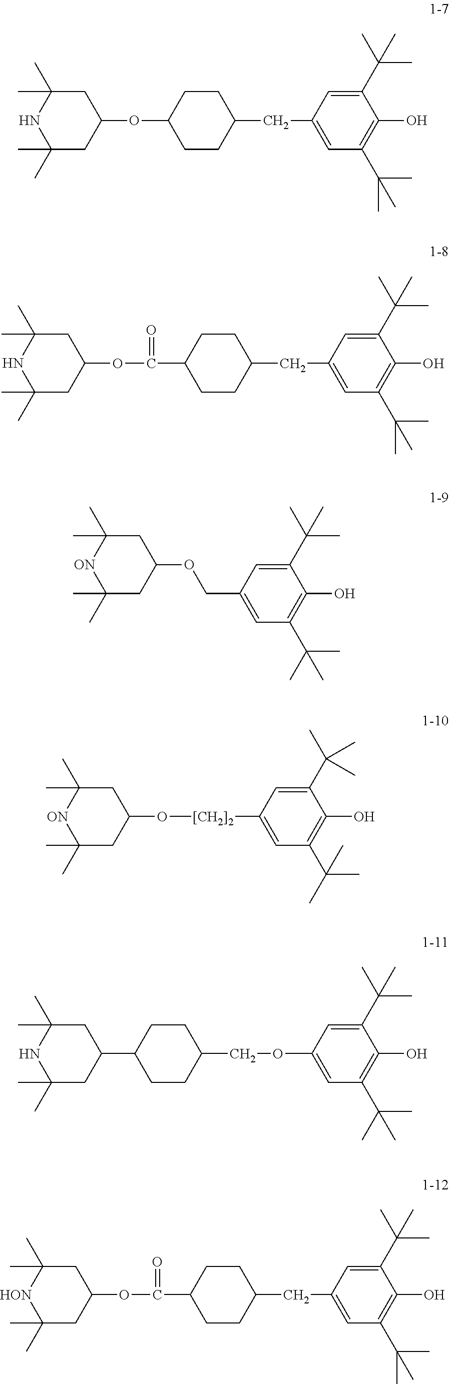 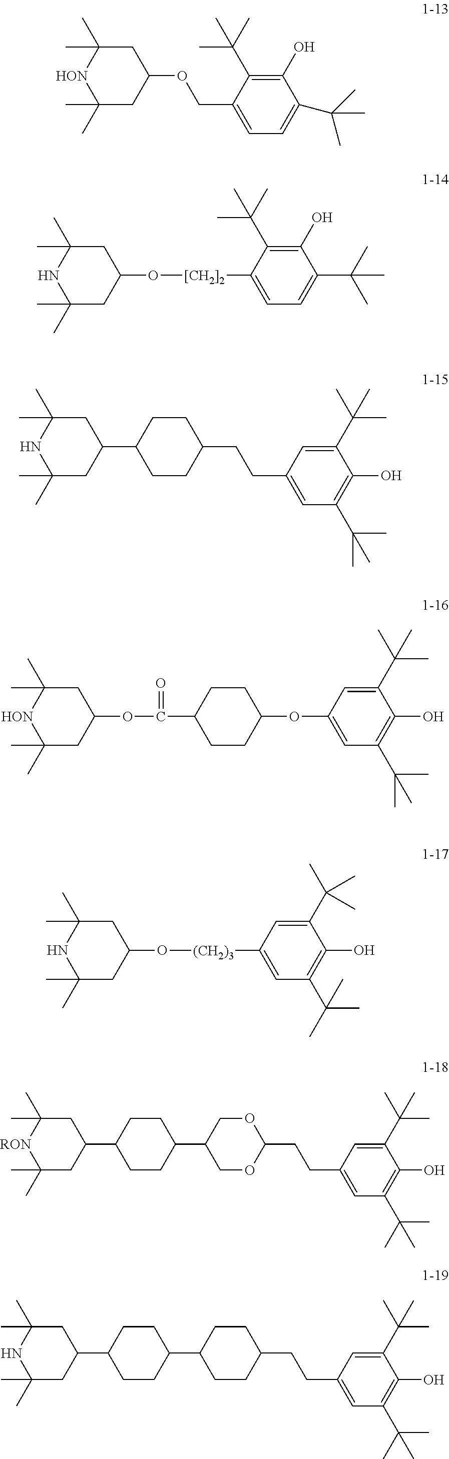 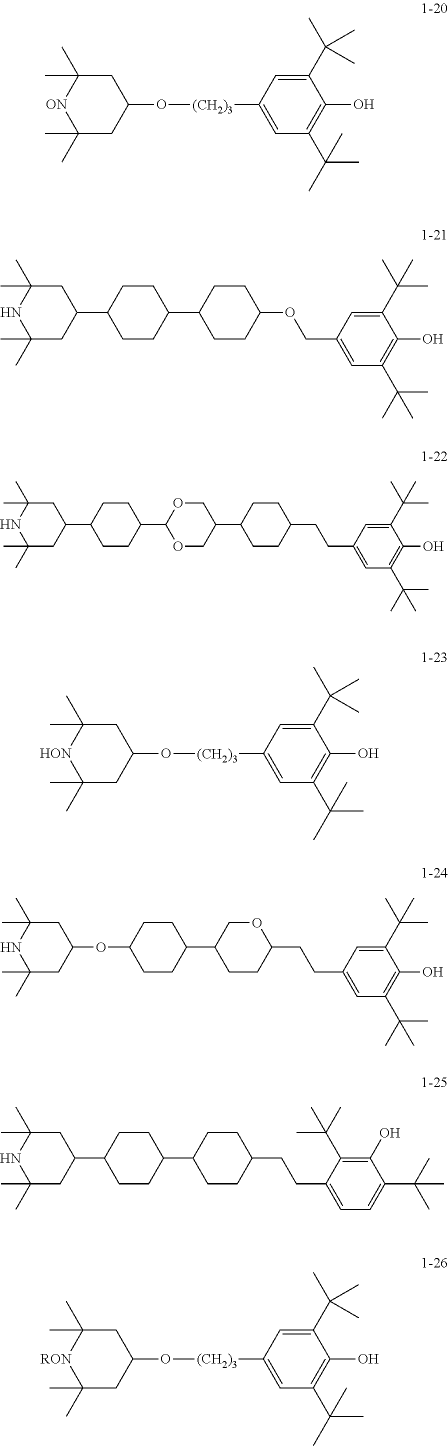 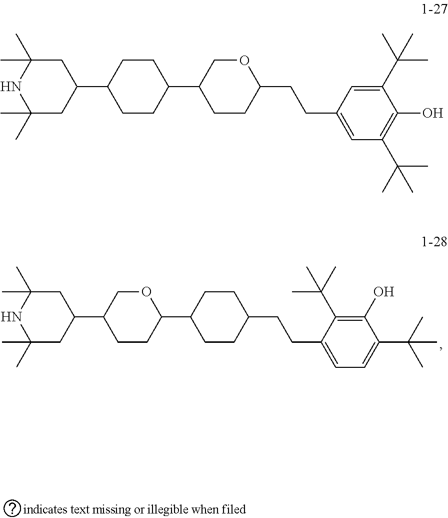
wherein R is an alkyl group having 1 to 5 carbon atoms.

12. The liquid crystal display of claim 9, wherein the compound represented by Chemical Formula 2 is at least one of the compounds represented by the following Chemical Formulae 2-1 to 2-16: 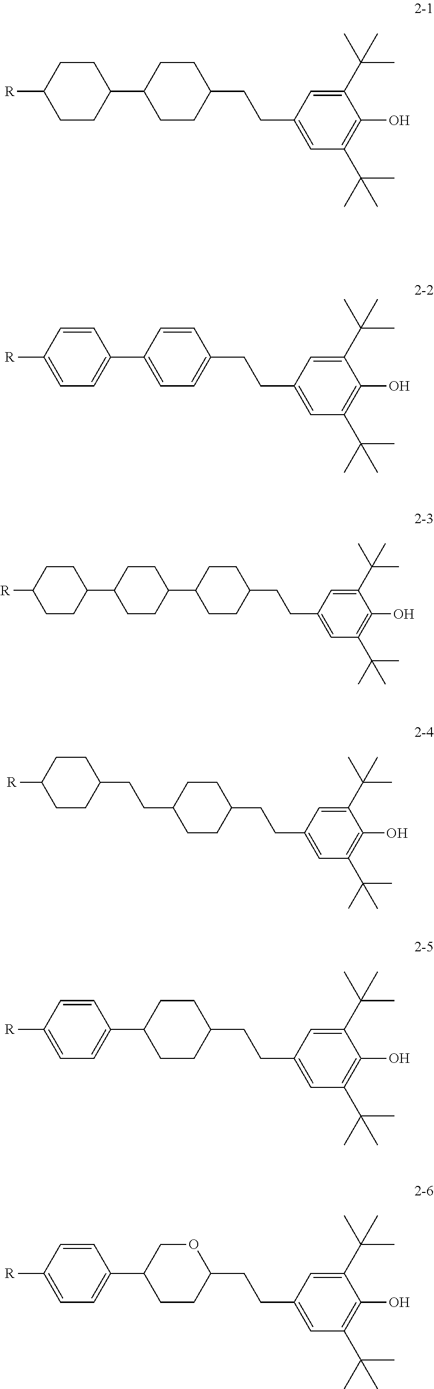 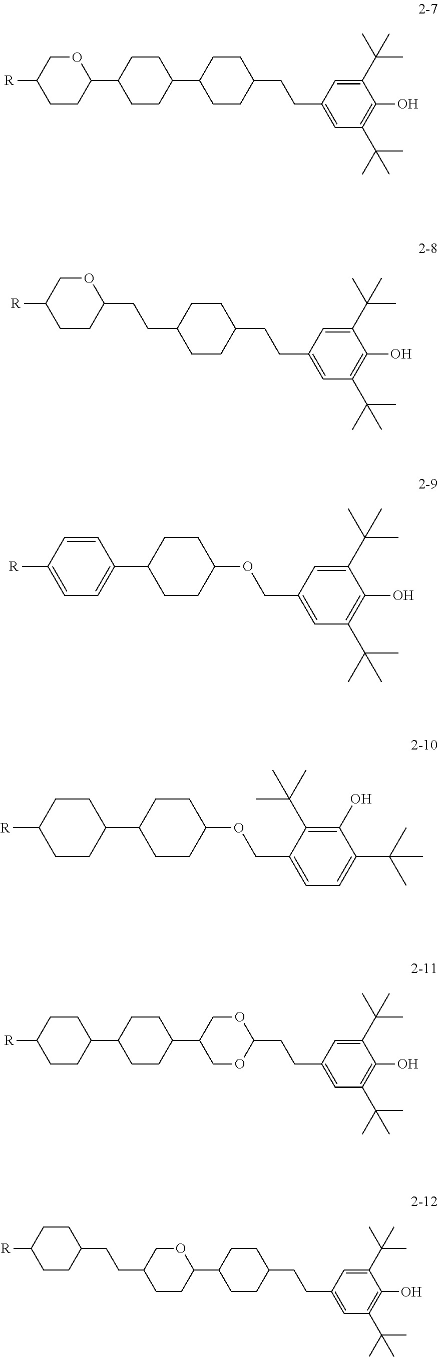 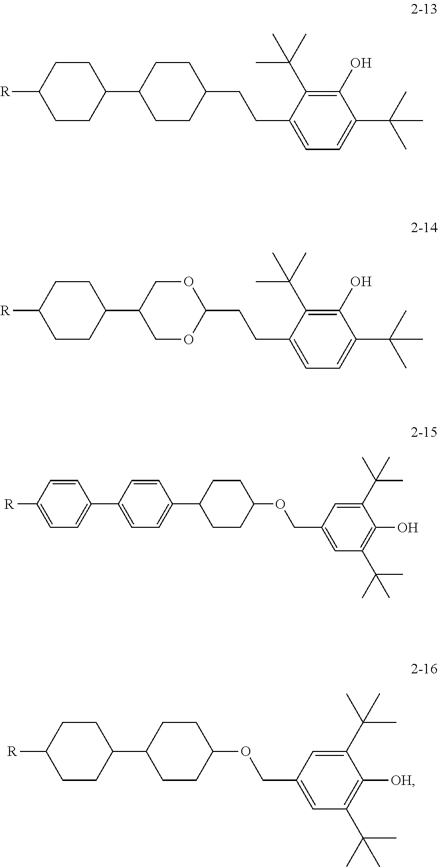
wherein R is an alkyl group having 1 to 5 carbon atoms.

13. The liquid crystal display of claim 9, wherein the first class of compounds includes at least one of the compounds represented by the following Chemical Formulae 3-1 to 3-16: 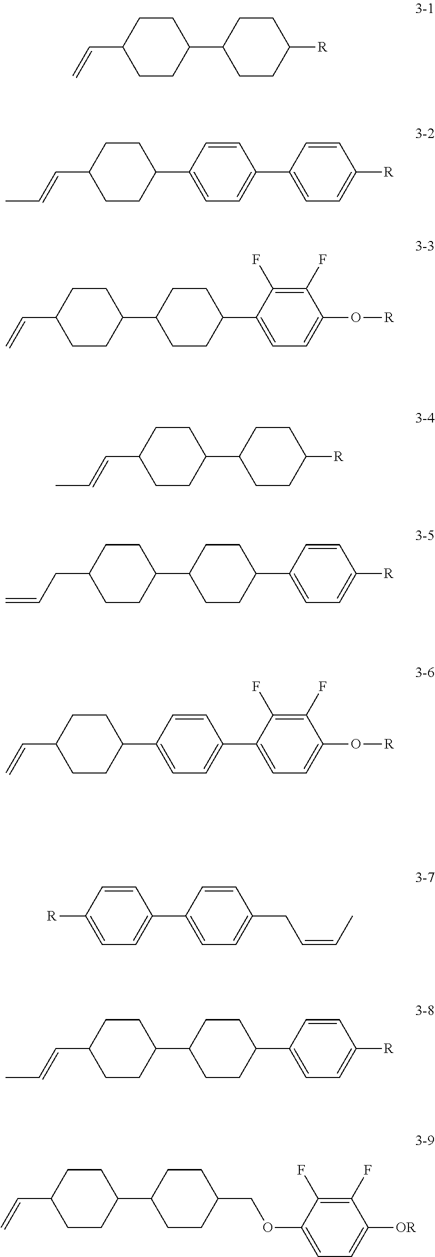 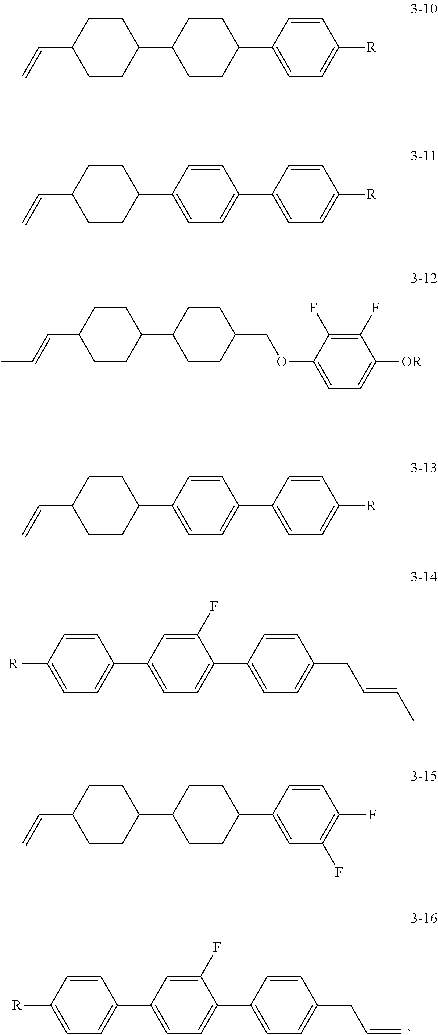
wherein R is an alkyl group or an alkoxy group having 1 to 5 carbon atoms.

14. A liquid crystal display comprising:
a first insulation substrate;
a pixel electrode disposed on the first insulation substrate;
a common electrode disposed on the first insulation substrate, wherein the common electrode is insulated from the pixel electrode;
a second insulation substrate facing the first insulation; and
a liquid crystal layer disposed between the first insulation substrate and the second insulation substrate,
wherein the liquid crystal layer includes a liquid crystal composition comprising:
a first class of compounds including an alkenyl group, and
a liquid crystal stabilizer including at least one of the compounds represented by the following Chemical Formulae 1 and 2: 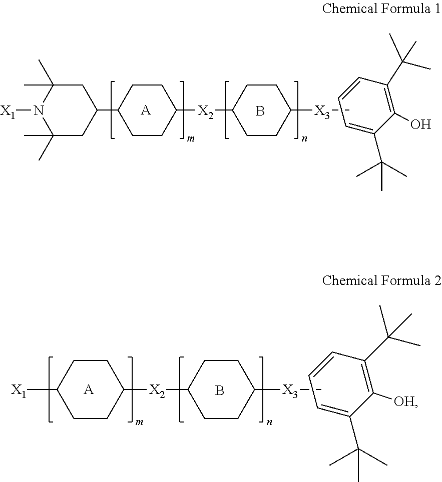
wherein X₁ is any one of H, O, OR, and R, X₂ is any one of O, OCO, OR, RO, NOR, and R, X₃ is any one of O, OCO, OR, RO, NOR, and R, A and B each independently include one or more of a cyclohexyl group, a cyclic ether group, and a phenyl group, R is an alkyl group having 1 to 5 carbon atoms, and m and n are each independently a natural number from 0 to 2.

15. The liquid crystal display of claim 14, wherein the liquid crystal layer includes the liquid crystal stabilizer in a content of from about 0.0001 wt % to about 10 wt % based on the liquid crystal layer.

16. The liquid crystal display of claim 14, wherein the compound represented by Chemical Formula 1 is at least one of the compounds represented by the following Chemical Formulae 1-1 to 1-28: 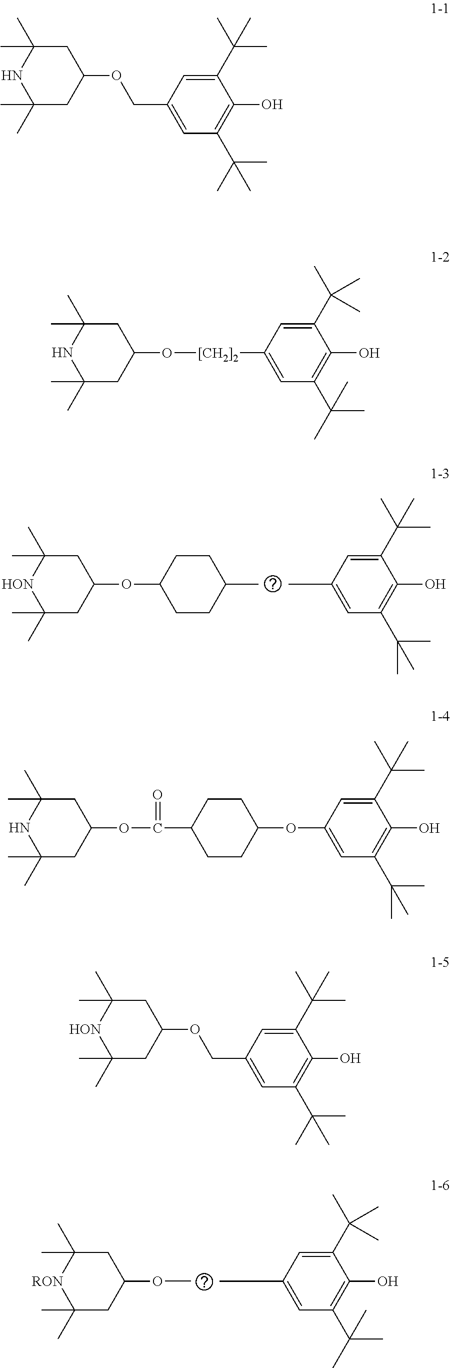 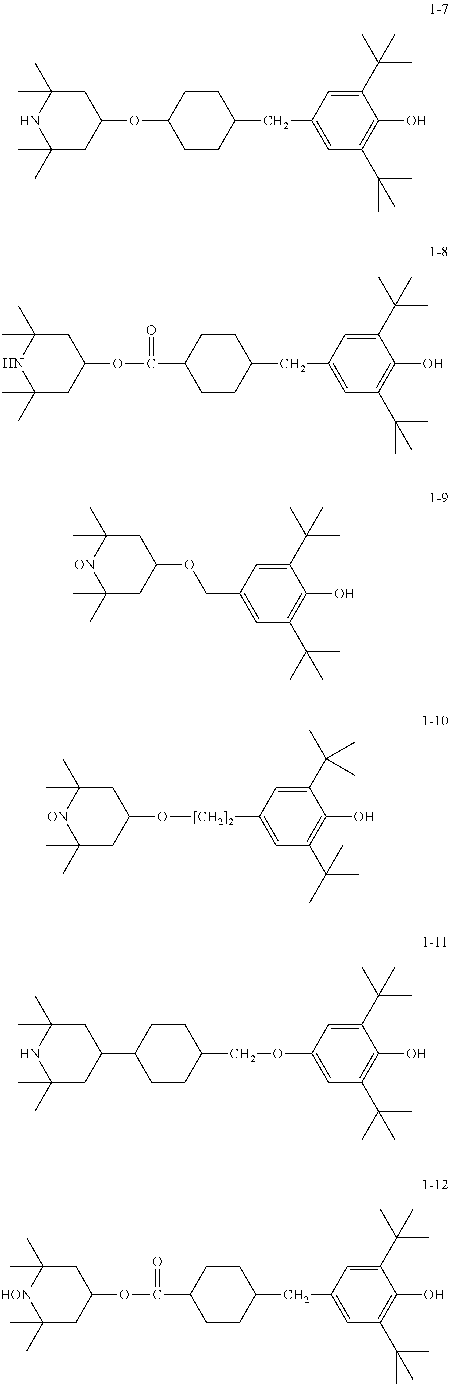 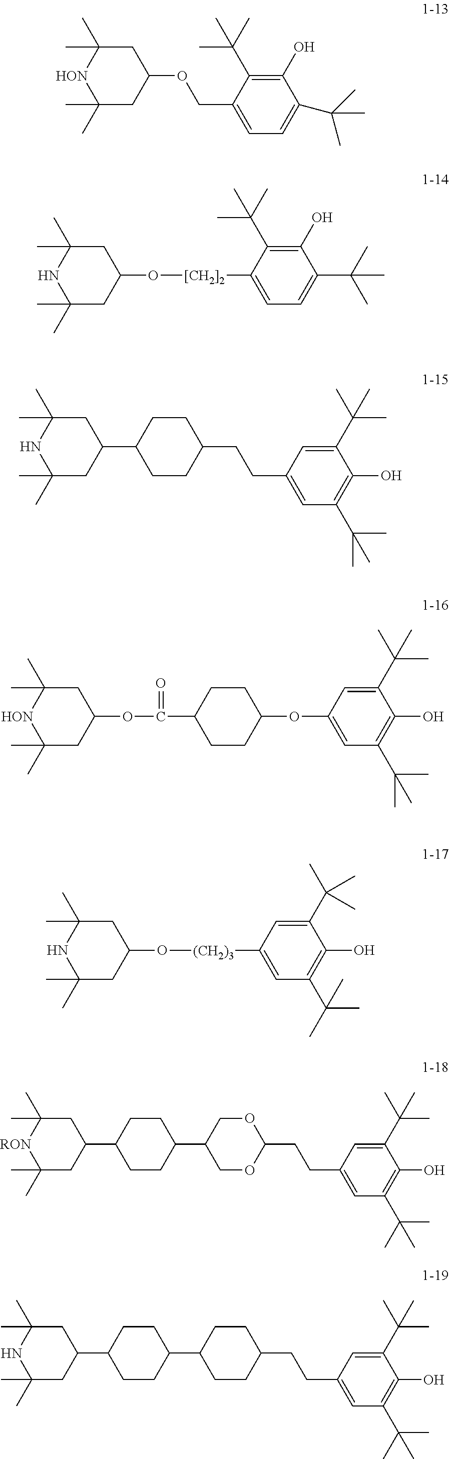 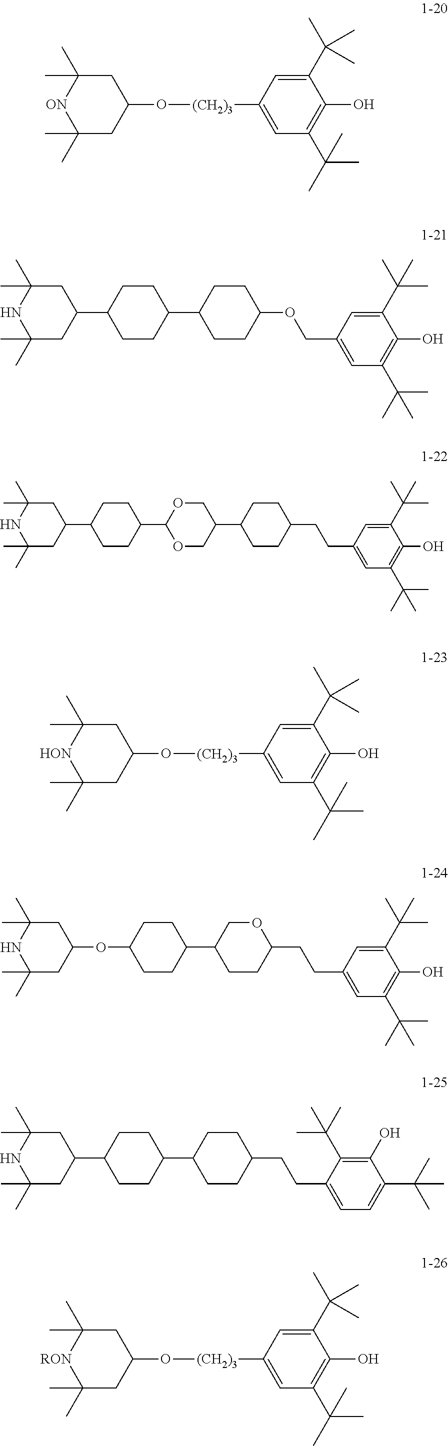 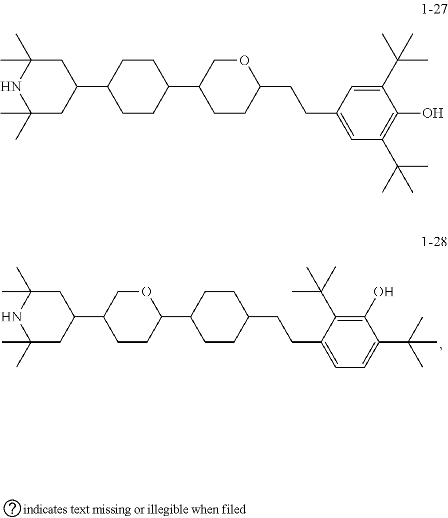
wherein R is an alkyl group having 1 to 5 carbon atoms.

17. The liquid crystal display of claim 14, wherein the compound represented by Chemical Formula 2 is at least one of the compounds represented by the following Chemical Formulae 2-1 to 2-16: 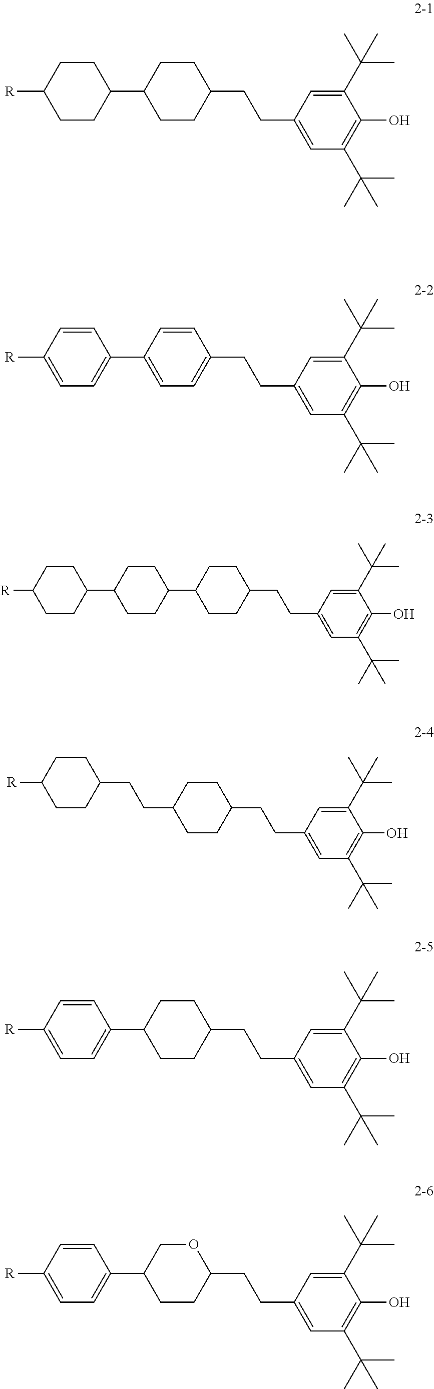 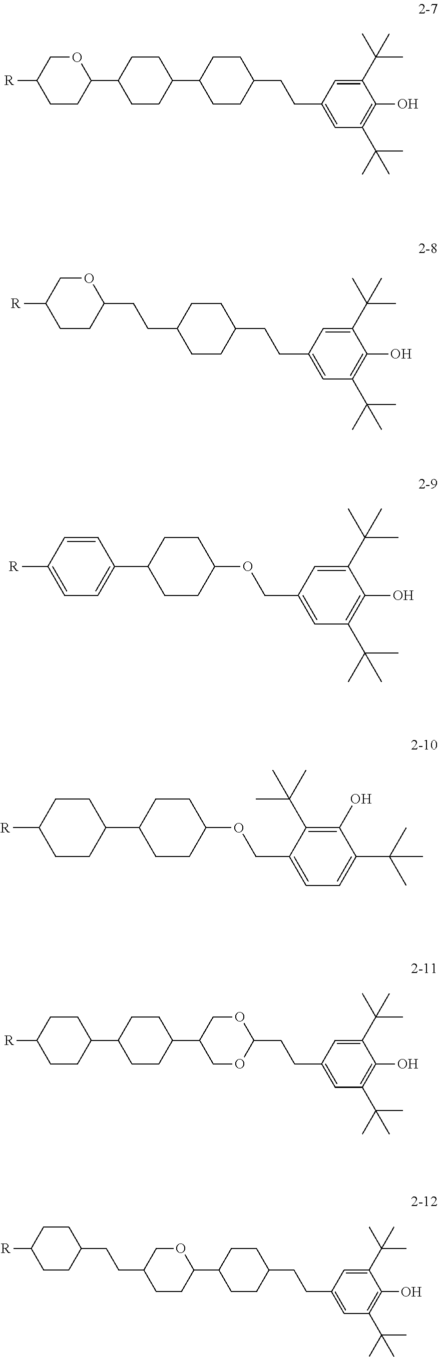 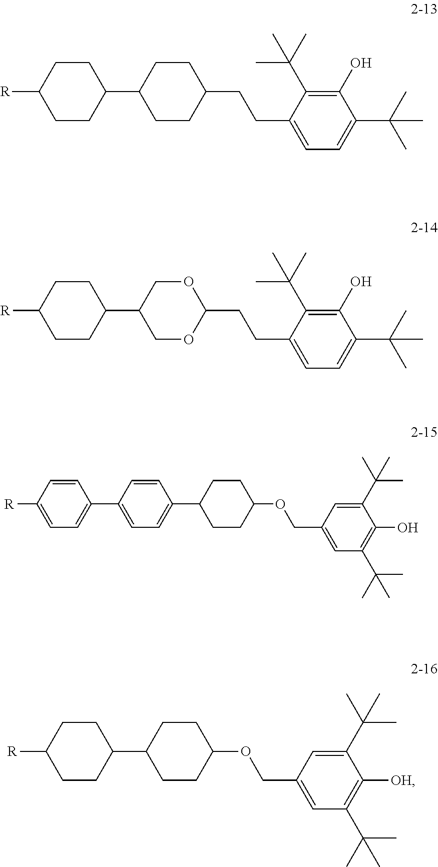
wherein R is an alkyl group having 1 to 5 carbon atoms.

18. The liquid crystal display of claim 14, wherein the first class of compounds includes at least one of the compounds represented by the following Chemical Formulae 3-1 to 3-16: 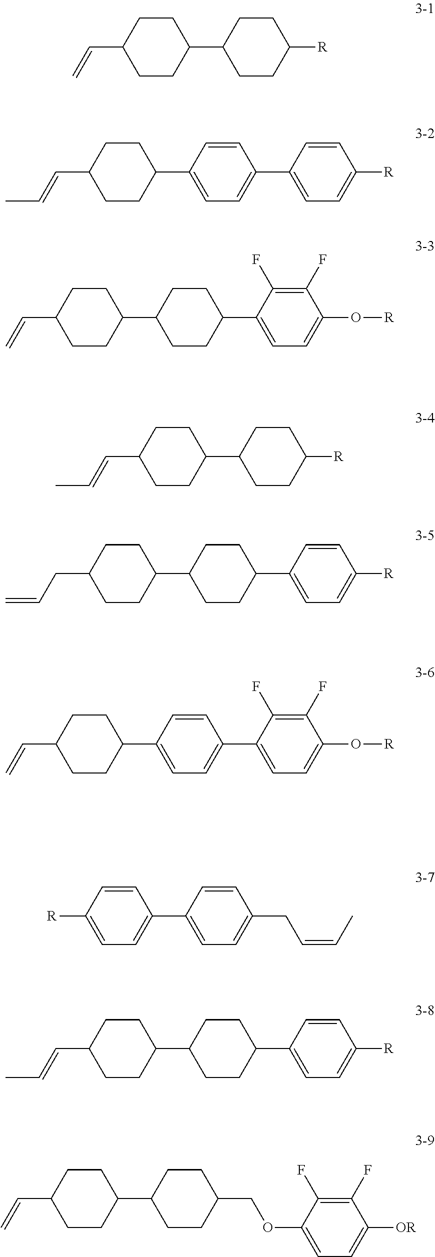 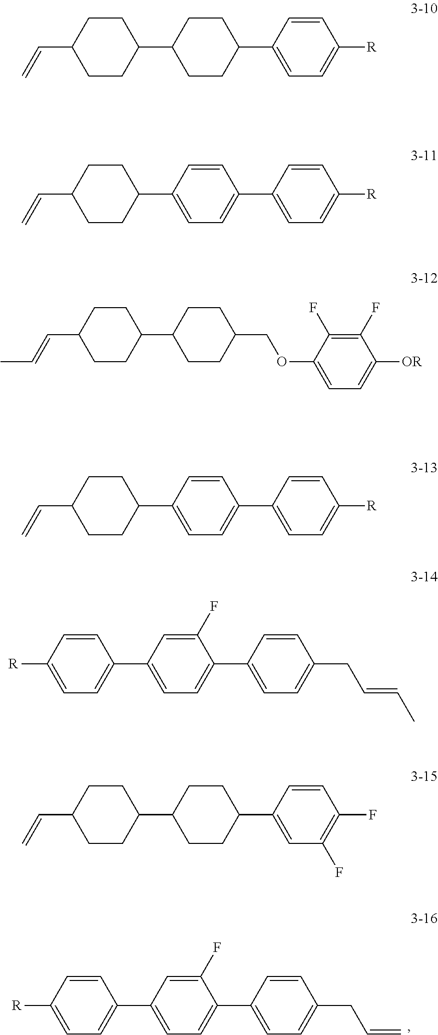
wherein R is an alkyl group or an alkoxy group having 1 to 5 carbon atoms.
